(19) United States
(12) Patent Application Publication
(10) Pub. No.: US 2025/0375424 A1
(43) Pub. Date: Dec. 11, 2025

(54) TINOSTAMUSTINE FOR USE IN TREATING SARCOMA

(71) Applicant: Purdue Pharma L.P., Stamford, CT (US)

(72) Inventor: Thomas Jorg Mehrling, Basel (CH)

(21) Appl. No.: 19/066,910

(22) Filed: Feb. 28, 2025

Related U.S. Application Data
(63) Continuation of application No. 18/108,736, filed on Feb. 13, 2023, now Pat. No. 12,257,237.
Application No. 18/108,736 is a continuation of application No. 16/621,885, filed on Dec. 12, 2019, now Pat. No. 11,576,899.

(30) Foreign Application Priority Data
Jun. 13, 2017 (GB) 1709403.8

(51) Int. Cl. A61K 31/4184; A61N 5/10; A61P 35/00

(52) U.S. Cl. CPC A61K 31/4184; A61N 5/10; A61P 35/00

(57) ABSTRACT

There is provided tinostamustine or a pharmaceutically acceptable salt thereof for use in the treatment of sarcoma in a patient in need thereof.

### CROSS-REFERENCE TO RELATED APPLICATIONS

This application is a continuation application of U.S. patent application Ser. No. 18/108,736, filed on Feb. 13, 2023, which is a continuation application of U.S. patent application Ser. No. 16/621,885, filed on Dec. 12, 2019, now U.S. Pat. No. 11,576,899, which is a U.S. national stage application filed under 35 U.S.C. 371 based on International Patent Application No. PCT/EP2018/065668, filed on Jun. 13, 2018, which claims priority to UK Patent Application No. GB1709403.8, filed on Jun. 13, 2017. The contents of each of the aforementioned applications are herein incorporated by reference in their entities.

### TECHNICAL FIELD

The present invention relates to methods of treating cancer, particularly sarcomas.

### BACKGROUND TO THE INVENTION

Cancer is one of the most life threatening diseases. Cancer is a condition in which cells in a part of the body experience out-of-control growth. According to latest data from American Cancer Society, it is estimated there will be 1.69 million new cases of cancer in USA in 2017. Cancer is the second leading cause of death in the United States (second only to heart disease) and will claim more than 601,000 lives in 2017. In fact, it is estimated the average lifetime risk of developing cancer is 40.8% for American males and 37.5% for American women. Therefore cancer constitutes a major public health burden and represents a significant cost in the United States. These figures are reflected elsewhere across most countries globally, although the types of cancer and relative proportions of the population developing the cancers vary depending upon many different factors such including genetics and diet.

For decades surgery, chemotherapy, and radiation were the established treatments for various cancers. Patients usually receive a combination of these treatments depending upon the type and extent of their disease. But chemotherapy is the most important option for cancer patients when surgical treatment (i.e. the removal of diseased tissue) is impossible. While surgery is sometimes effective in removing tumours located at certain sites, for example, in the breast, colon, and skin, it cannot be used in the treatment of tumours located in other areas, such as the backbone, nor in the treatment of disseminated hematological cancers including cancers of the blood and blood-forming tissues (such as the bone marrow). Such cancers include multiple myeloma, lymphoma and leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. It is used most often in the treatment of breast, lung, and testicular cancer. One of the main causes of failure in chemotherapy is the development of drug resistance by the cancer cells, a serious problem that may lead to recurrence of disease or even death. Thus, more effective cancer treatments are needed.

Solid tumours are an abnormal mass of tissue that usually does not contain cysts or liquid areas. Solid tumours may be benign (not cancer), or malignant (cancer). Different types of solid tumours are named for the type of cells that form them. Examples of solid tumours are carcinomas and sarcomas. The four most common cancers occurring worldwide are all solid tumours, namely lung, breast, bowel and prostate cancer. These four solid tumour cancers account for around 4 in 10 of all cancers diagnosed worldwide.

However, not all solid tumours are as common. Sarcomas are rare cancers that can develop in almost any part of the body, including muscle, bone, nerves, cartilage, tendons, blood vessels and the fatty and fibrous tissues. There are three main types of sarcoma: soft tissue sarcoma, bone sarcoma and gastrointestinal stromal tumours (GIST) and in the US, around 20,000 new sarcomas are diagnosed each year. The overall relative 5-year survival rate of people with soft tissue sarcomas is around 50% according to statistics from the National Cancer Institute (NCI).

There is therefore a need for new effective chemotherapeutic treatments.

In WO-A-2010/085377, the compound of formula I below is disclosed. It is a first-in-class dual-functional alkylating-HDACi fusion molecule which potently inhibits HDAC-regulated pathways.

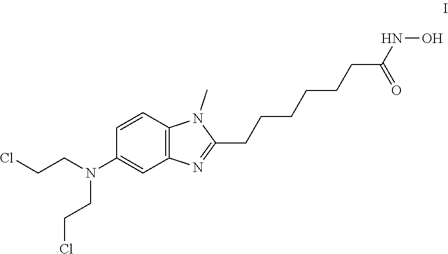 [SMILES: Cn1c(CCCCCCC(=O)NO)nc2cc(N(CCCl)CCCl)ccc21]

Biological assays showed that the compound of formula I potently inhibits HDAC enzyme (HDAC1 IC₅₀ of 9 nM). The compound of formula I has an INN of tinostamustine and is also known in the art as EDO-S101. It is an AK-DAC (a first-in-class alkylating deacetylase molecule) that, in preclinical studies, has been shown to simultaneously improve access to the DNA strands within cancer cells, break them and block damage repair.

### SUMMARY OF THE INVENTION

In a first aspect of the present invention there is provided tinostamustine or a pharmaceutically acceptable salt thereof for use in the treatment of sarcoma in a patient in need thereof selected from soft tissue sarcoma, bone sarcoma or non-KIT gastrointestinal stromal tumour (GIST).

It has surprisingly been discovered that tinostamustine or a pharmaceutically acceptable salt thereof is particularly effective in the treatment of sarcoma, with activity data showing strong sensitivity to this compound. Thus, the need for a new and effective treatment of sarcoma is met by the present invention.

In a further aspect of the present invention there is provided the use of tinostamustine or a pharmaceutically acceptable salt thereof in the manufacture of a medicament for the treatment of sarcoma selected from soft tissue sarcoma, bone sarcoma or non-KIT gastrointestinal stromal tumour (GIST).

In a further aspect of the present invention there is provided a method of treating sarcoma selected from soft tissue sarcoma, bone sarcoma or non-KIT gastrointestinal stromal tumour (GIST), in a patient in need thereof comprising administering to said patient an effective amount of tinostamustine or a pharmaceutically acceptable salt thereof.

In a further aspect of the present invention there is provided a kit comprising tinostamustine or a pharmaceutically acceptable salt thereof together with instructions for treating sarcoma selected from soft tissue sarcoma, bone sarcoma or non-KIT gastrointestinal stromal tumour (GIST).

The following features apply to all aspects of the invention.

The sarcoma may be a soft tissue sarcoma.

The sarcoma may be a bone sarcoma.

The sarcoma may be from the Ewing family of tumours. The sarcoma may be Ewing tumour of bone. The sarcoma may be an extraosseous Ewing tumour. The sarcoma may start in the bone.

The sarcoma may start in the soft tissue. The sarcoma may be primitive neuroectodermal tumour (PNET).

The sarcoma may be liposarcoma. The liposarcoma may be well-differentiated liposarcoma.

The liposarcoma may be myxoid liposarcoma. The liposarcoma may be pleomorphic liposarcoma. The liposarcoma may be dedifferentiated liposarcoma. Preferably the liposarcoma may be dedifferentiated liposarcoma.

The sarcoma may be non-KIT GIST.

The sarcoma may be wildtype GIST.

The sarcoma may be paediatric GIST.

The sarcoma may be relapsed and/or refractory.

The sarcoma may be localized.

The sarcoma may be metastatic.

The sarcoma may be advanced.

The sarcoma may have progressed after at least one line of standard therapy.

### DESCRIPTION OF THE DRAWINGS

FIG. 1 shows the antitumoural activity of EDO-S101 against TC-71 Ewing sarcoma.

FIG. 2 shows the antitumoural activity of EDO-S101 against DD013 dedifferentiated liposarcoma sarcoma.

### DETAILED DESCRIPTION OF THE INVENTION

In the present application, a number of general terms and phrases are used, which should be interpreted as follows.

The compound of formula I has an INN of tinostamustine and is also known in the art as EDO-S101. The IUPAC name is 7-(5-(bis(2-chloroethyl)amino)-1-methyl-1H-benzo[d]imidazol-2-yl)-N-hydroxyheptanamide.

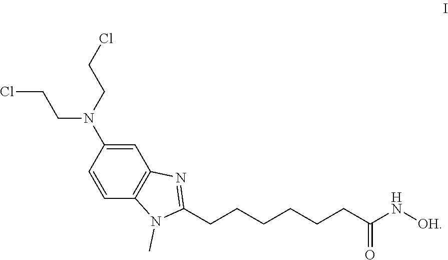 [SMILES: Cn1c(CCCCCCC(=O)NO)nc2cc(N(CCCl)CCCl)ccc21]

“Patient” includes humans, non-human mammals (e.g., dogs, cats, rabbits, cattle, horses, sheep, goats, swine, deer, and the like) and non-mammals (e.g., birds, and the like).

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In the present invention, the pharmaceutically acceptable salt of tinostamustine may preferably be the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate salt.

It has surprisingly been found that tinostamustine or a pharmaceutically acceptable salt thereof shows surprising efficacy in solid tumours. In particular, it has been found that tinostamustine or a pharmaceutically acceptable salt thereof is useful in the treatment of sarcomas.

Sarcomas are rare cancers that develop in the muscle, bone, nerves, cartilage, tendons, blood vessels and the fatty and fibrous tissues. They can affect almost any part of the body, on the inside or the outside. Sarcomas commonly affect the arms, legs and trunk. They also appear in the stomach and intestines as well as behind the abdomen (retroperitoneal sarcomas) and the female reproductive system (gynaecological sarcomas).

Bone sarcomas affect less than 500 people in the UK each year, making it a very rare form of cancer. Not all bone cancers will be sarcomas.

Soft tissue sarcomas can affect any part of the body. They develop in supporting or connective tissue such as the muscle, nerves, fatty tissue, and blood vessels. Soft tissue sarcomas include: GIST which is a common type of sarcoma which develops in the gastrointestinal (GI) tract; gynaecological sarcomas which occur in the female reproductive system: the uterus (womb), ovaries, vagina, vulva and fallopian tubes; and retroperitoneal sarcomas which occur in the retroperitoneum.

Unless detected at an early stage when the tumour can be removed by surgery there is currently no cure for soft tissue sarcoma. Approximately 16% of patients with soft tissue sarcoma have advanced stage (metastatic) disease. For these patients, the relative 5 year survival rate is 16% (American Cancer Society).

### Liposarcoma

One particular soft tissue sarcoma is liposarcoma. Liposarcoma is a rare cancer of connective tissues that resemble fat cells under a microscope. It accounts for up to 18% of all soft tissue sarcomas. Liposarcoma can occur in almost any part of the body, but more than half of liposarcoma cases involve the thigh, and up to a third involve the abdominal cavity. Liposarcoma tends to affects adults between the ages of 40 and 60. When it does occur in children, it is usually during the teenage years. There are four types of liposarcoma as shown below. The risk of recurrence and metastasis with liposarcoma increases with higher grade.

Well-differentiated liposarcoma is the most common subtype and usually starts as a low grade tumour. Low grade tumour cells look much like normal fat cells under the microscope and tend to grow and change slowly.

Myxoid liposarcoma is an intermediate to high grade tumour. Its cells look less normal under the microscope and may have a high grade component.

Pleomorphic liposarcoma is the rarest subtype and is a high grade tumour with cells that look very different from normal cells.

Dedifferentiated liposarcoma occurs when a low grade tumour changes, and the newer cells in the tumour are high grade.

### Ewing Family of Tumours

The Ewing family of tumours is a group of cancers that start in the bones or nearby soft tissues that share some common features. These tumours can develop at any age, but they are most common in the early teen years. The main types of Ewing tumours are:
Ewing sarcoma of bone: Ewing sarcoma that starts in a bone is the most common tumour in this family. This type of tumour was first described by Dr. James Ewing in 1921, who found it was different from the more common bone tumour, osteosarcoma. Seen under a microscope, its cells looked different from osteosarcoma cells. It was also more likely to respond to radiation therapy.
Extraosseous Ewing tumour (EOE): Extraosseous Ewing tumours start in soft tissues around bones, but they look and act very much like Ewing sarcomas in bones. They are also known as extraskeletal Ewing sarcomas.
Primitive neuroectodermal tumour (PNET): This rare childhood cancer also starts in bone or soft tissue and shares many features with Ewing sarcoma of bone and EOE. PNETs that start in the chest wall are known as Askin tumours. PNETs that start in the bone are known as peripheral neuroectodermal sarcoma of bone.

The cells that make up Ewing sarcoma, EOE, and PNET are very similar. They tend to have the same DNA (gene) abnormalities and share similar proteins, which are rarely found in other types of cancers. The three cancers are thought to develop from the same type of cells and while there are differences among these tumours, they are all currently treated in the same way.

Most Ewing tumours occur in the bones. The most common sites are: the pelvis (hip bones), the chest wall (such as the ribs or shoulder blades), or the legs, mainly in the middle of the long bones. Extraosseous Ewing tumours can occur almost anywhere.

Most Ewing tumours occur in children and teens, but they can also occur in adults.

### Gastrointestinal Stromal Tumours (GISTs)

A further type of soft tissue sarcomas are gastrointestinal stromal tumours (GISTs). GISTs are uncommon tumours of the gastrointestinal (GI) tract. Although they comprise fewer than 1% of all GI tumours, GIST are the most common mesenchymal tumours of the GI tract. According to the National Cancer Institute, it has been estimated that there are 3,300 to 6,000 new GIST cases per year in the United States. A study based on Surveillance, Epidemiology and End Results (SEER) registry data found that the age-adjusted yearly incidence of GIST in the United States was 6.8 per million from 1992 to 2000. However, the true incidence is not known, in part because many tumours have not been tested for the characteristic KIT (CD117) or platelet-derived growth factor receptor alpha (PDGFRA) gene mutations. In addition, small, indolent GIST, only a few millimeters in diameter, are common in the general population and are not included in cancer registries. GIST are equally distributed across all geographic and ethnic groups and men and women are equally affected. Most patients present between the ages of 50 and 80. The vast majority of GIST are sporadic, but there are rare familial forms associated with the characteristic heritable mutations in the KIT gene (or, rarely, in succinate dehydrogenase genes in Carney-Stratakis syndrome). Familial GIST may present as multiple primary tumours.

GIST can occur anywhere along the GI tract, but most often are found in the stomach or small intestine. The American Joint Committee on Cancer (AJCC) Cancer Staging Manual lists the following approximate distributions: stomach (60%); small intestine (30%); rectum (3%); colon (1-2%); esophagus (<1%); omentum/mesentery (rare).

Less frequently, GIST may arise in the appendix, gallbladder, pancreas, retroperitoneum, and paravaginal and periprostatic tissues. Approximately 20% to 25% of gastric GIST and 40% to 50% of small intestinal GIST are clinically aggressive. It has been estimated that approximately 10% to 25% of patients present with metastatic disease.

The clinical presentation of patients with GIST varies depending on the anatomic location of the tumour and the tumour size and aggressiveness. The most common presentation of GIST is GI bleeding, which may be acute (melena or hematemesis) or chronic and results in anemia.

Smaller lesions may be incidental findings during surgery, radiologic studies, or endoscopy. The natural history of these incidental tumours and the frequency of progression to symptomatic disease are unknown. There may be a substantial reservoir of small GIST tumours that do not progress to symptomatic stages. For example, a series of 98 consecutive systematic autopsies on adults who died of unrelated causes revealed grossly recognizable gastric tumours (1 mm-6 mm) that were histologically diagnosed as GIST in 22.5% of cases. Sufficient DNA was available for analysis in 26 patients, revealing 13 patients with mutations in KIT exon 11 and one in PDGFRA.

In a retrospective study of 200 GIST cases, typical clinical manifestations of malignancy included liver metastases and/or dissemination within the abdominal cavity. Lymph node involvement and spread to the lungs or other extra-abdominal sites was unusual. Advanced disease may be associated with metastases to distant sites, including lung and bone. Brain metastases are rare.

Typically arising within the muscle wall of the GI tract, GIST range in size from less than 1 cm to more than 40 cm, with an average size of approximately 5 cm when diagnosed clinically. [2] Small GIST may form solid subserosal, intramural, or, less frequently, polypoid intraluminal masses. Large tumours tend to form external masses attached to the outer aspect of the gut involving the muscular layers. GIST morphology is quite varied; the tumours are composed of the following: spindle cells (70%); epithelioid cells (20%); and mixed spindle and epithelioid cells (10%).

GIST encompass a broad continuum of histologic patterns, ranging from bland-appearing tumours with very low mitotic activity (often previously designated leiomyomas) to very aggressive-appearing patterns (previously often called leiomyosarcomas). They may originate from interstitial cells of Cajal (ICC) or their stem cell-like precursors, although this is not certain.

The most commonly used marker for GIST is the CD117 antigen, a marker expressed by ICC. Approximately 95% of GISTs are positive for the CD117 antigen, an epitope of the KIT receptor tyrosine kinase. However, CD117 immunohistochemistry is not specific for GIST, as weak reactivity occurs with other mesenchymal neoplasms; accordingly, morphologic examination and the use of other immunostains in difficult cases are indispensable. In addition, false-positive CD117 staining can occur if antigen retrieval techniques are used in the pathology laboratory to enhance marker expression.

Approximately 85% of GIST contain oncogenic mutations in one of two receptor tyrosine kinases: KIT or PDGFRA (platelet-derived growth factor receptor alpha). Constitutive activation of either of these receptor tyrosine kinases plays a central role in the pathogenesis of GIST. Wild-type tumours, with no detectable KIT or PDGFRA mutations, account for 12% to 15% of all GIST. Fewer than 5% of GIST occur in the setting of syndromic diseases, such as neurofibromatosis type 1 (NF1), Carney triad syndrome, and other familial diseases. The correct identification of GIST is important because of the availability of specific, molecular-targeted therapy with KIT/PDGFRA tyrosine kinase inhibitors (TKI) such as imatinib mesylate or, in the case of imatinib-resistant GIST, sunitinib malate.

Wild-type or non-KIT GIST represents an even narrower sub-set of GIST patients that do not respond as well to current therapies. As discussed above, GIST typically presents in patients aged 50 to 80. Pediatric GIST is therefore rare but it has been suggested that the biology of pediatric GIST is different from adult GIST. For example, patients with pediatric GIST are predominantly female, their tumours are of epithelioid or mixed histology, they lack large-scale chromosomal aberrations, and only rarely (<15%) do they have KIT or PDGFRA mutations. As such, paediatric GIST typically is non-KIT.

Compared to other intra-abdominal sarcomas, survival in GIST patients after surgery alone is favorable. In a retrospective study involving 200 patients that predated the use of TKI, the 5-year disease-specific survival rate for GIST patients with primary disease who underwent complete resection of gross disease (N=80) was 54%, with survival predicted by tumour size; the overall disease-specific survival was 35% at 5 years. Other studies, which also predated TKI, reported 5-year survival rates of 40% to 63% for patients undergoing complete resections of GIST.

The median disease-specific survival of patients with metastatic GIST (N=94) was 19 months. In one retrospective study involving 119 patients with metastatic GIST, it was found that once a GIST becomes metastatic, kinase genotype did not factor into overall survival. The median time to recurrence for patients on imatinib is 2 years.

The therapeutically effective amount of tinostamustine or a pharmaceutically acceptable salt administered to the patient is an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of tinostamustine or a pharmaceutically acceptable salt thereof according to the present invention is believed to be one wherein tinostamustine or a pharmaceutically acceptable salt thereof is included at a dosage range of from 0.3 mg/m² to 300 mg/m² body surface area of the patient or from 60 mg/m² to 150 mg/m² body surface area of the patient. In a preferred embodiment, the dosage range is from 80 to 100 mg/m² body surface area of the patient.

The specific therapeutically effective dose level for any particular patient will depend upon a variety of factors including the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

“Metastatic Cancer”. Cancer has the ability to spread within the body. Cancer cells can spread locally by moving into nearby normal tissue. Cancer can also spread regionally, to nearby lymph nodes, tissues, or organs. Cancer can therefore spread to distant parts of the body. When this happens, it is called metastatic cancer (also known as stage IV cancer), and the process by which cancer cells spread to other parts of the body is called metastasis. Thus, in metastasis, cancer cells break away from where they first formed (primary cancer), travel through the blood or lymph system, and form new tumours (metastatic tumours) in other parts of the body.

Metastatic cancer cells have features like that of the primary cancer and not like the cells in the place where the cancer is found. This enables doctors to tell whether a cancer is metastatic. Metastatic cancers are given the same name as the primary cancer. For example, breast cancer that has spread to the lung is called metastatic breast cancer, not lung cancer. It is treated as stage IV breast cancer, not as lung cancer.

Metastatic sarcoma (for example metastatic soft tissue sarcoma, metastatic bone sarcoma or metastatic non-KIT gastrointestinal stromal tumour (GIST) refers to a sarcoma that has metastasised to a new location in the body. The cancer is treated as a stage IV sarcoma (for example a stage IV soft tissue sarcoma, a stage IV bone sarcoma or stage IV non-KIT GIST).

“Advanced Cancer” is a cancer that is not curable but responds to treatment. Disease directed therapy is still very important because it prolongs life. For terminal cancer, therapy cannot prolong survival significantly due to the progressive nature of the disease and palliative care is the main treatment option.

Suitable examples of the administration form of tinostamustine or a pharmaceutically acceptable salt thereof include without limitation oral, topical, parenteral, sublingual, rectal, vaginal, ocular, and intranasal. Parenteral administration includes subcutaneous injections, intravenous, intramuscular, intrasternal injection or infusion techniques. Preferably, tinostamustine or a pharmaceutically acceptable salt thereof is administered parenterally, and most preferably intravenously.

Preferably, tinostamustine or a pharmaceutically acceptable salt thereof is administered intravenously to the patient in need thereof at a dosage level to the patient in need thereof of from 0.3 mg/m² to 300 mg/m² body surface area of the patient.

Preferably, tinostamustine or a pharmaceutically acceptable salt thereof is administered intravenously to the patient in need thereof at a dosage level to the patient in need thereof of from 60 mg/m² to 150 mg/m² body surface area of the patient.

Preferably, tinostamustine or a pharmaceutically acceptable salt thereof is administered intravenously to the patient in need thereof at a dosage level to the patient in need thereof of from 80 mg/m² to 100 mg/m² body surface area of the patient.

It has been found that in embodiments of the present invention, tinostamustine or a pharmaceutically acceptable salt thereof or medicament comprising the same may preferably be administered to a patient in need thereof on days 1, 8 and 15 of a 28 day treatment cycle or on days 1 and 15 of a 28 day treatment cycle.

Preferably, tinostamustine or a pharmaceutically acceptable salt thereof is administered on days 1 and 15 of a 28 day treatment cycle.

It has been found that in embodiments of the present invention, tinostamustine or a pharmaceutically acceptable salt thereof or medicament comprising the same may preferably be administered to a patient in need thereof over an infusion time of 60 minutes; or an infusion time of 45 minutes; or an infusion time of 30 minutes.

Preferably, tinostamustine or a pharmaceutically acceptable salt thereof is administered over an infusion time of 60 minutes.

In a preferred embodiment, tinostamustine or a pharmaceutically acceptable salt is administered to the patient in need thereof at a dosage level of from 80 mg/m² to 100 mg/m² body surface area of the patient, on days 1 and 15 of a 28 day treatment cycle, over an infusion time of 60 minutes.

In embodiments of the present invention, there is provided a kit comprising tinostamustine or a pharmaceutically acceptable salt thereof or medicament comprising the same together with instructions.

The instructions may advise administering tinostamustine or a pharmaceutically acceptable salt thereof according to variables such as the state of the solid tumours being treated; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts.

In a further embodiment of the present invention, the patient in need of said treatment is given radiotherapy with (including prior to, during or after) treatment of the solid tumour(s) with tinostamustine or a pharmaceutically acceptable salt thereof. In embodiments of the present invention, the patient is treated with tinostamustine or a pharmaceutically acceptable salt thereof and radiotherapy. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

In a further embodiment of the present invention, the patient in need of said treatment is given radiotherapy prior to or after treatment of the solid tumours with tinostamustine or a pharmaceutically acceptable salt thereof. Preferably, the patient is given radiotherapy treatment prior to the treatment with tinostamustine or a pharmaceutically acceptable salt thereof. The radiotherapy may be given at a dose of 1 to 5 Gy over 5-10 consecutive days and preferably 2 Gy over 5-10 consecutive days.

When intended for oral administration, tinostamustine or a pharmaceutically acceptable salt thereof or medicament comprising the same may be in solid or liquid form, where semi-solid, semi-liquid, suspension and gel forms are included within the forms considered herein as either solid or liquid.

Tinostamustine or a pharmaceutically acceptable salt thereof or medicament comprising the same can be prepared for administration using methodology well known in the pharmaceutical art. Examples of suitable pharmaceutical formulations and carriers are described in “Remington's Pharmaceutical Sciences” by E. W. Martin.

As a solid composition for oral administration, tinostamustine or a pharmaceutically acceptable salt thereof can be formulated into a powder, granule, compressed tablet, pill, capsule, chewing gum, wafer or the like form. Such a solid composition typically contains one or more inert diluents or carriers. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When tinostamustine or a pharmaceutically acceptable salt thereof compositions is in the form of a capsule (e.g. a gelatin capsule), it can contain, in addition to materials of the above type, a liquid carrier such as polyethylene glycol, cyclodextrin or a fatty oil.

Tinostamustine or a pharmaceutically acceptable salt thereof compositions can be in the form of a liquid, e.g. an elixir, syrup, solution, emulsion or suspension. The liquid can be useful for oral administration or for delivery by injection. When intended for oral administration, tinostamustine or a pharmaceutically acceptable salt thereof compositions can comprise one or more of a sweetening agent, preservatives, dye/colorant and flavor enhancer. In tinostamustine or a pharmaceutically acceptable salt thereof compositions for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, tinostamustine or a pharmaceutically acceptable salt thereof or medicament comprising the same is administered intravenously.

Liquid forms of tinostamustine or a pharmaceutically acceptable salt thereof or medicament comprising the same, may be solutions, suspensions or other like form, and can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

Tinostamustine or a pharmaceutically acceptable salt thereof or medicament comprising the same can be administered by any convenient route, for example by infusion or bolus injection, by absorption through epithelial or mucocutaneous linings, and preferably by bolus.

Examples of compositions comprising tinostamustine or a pharmaceutically acceptable salt thereof are disclosed in WO2013/040286.

The present invention may be further understood by consideration of the following non-limiting examples.

### EXAMPLES

In the following examples, tinostamustine is referred to as EDO-S101 and has the following formula:

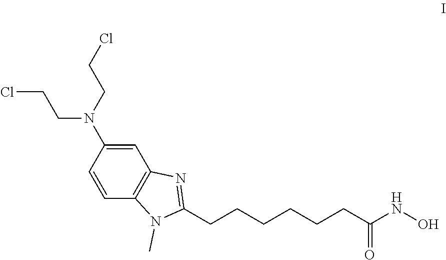 [SMILES: Cn1c(CCCCCCC(=O)NO)nc2cc(N(CCCl)CCCl)ccc21]

EDO-S101 may be prepared as described in Example 6 of WO-A-2010/085377.

### Materials and Methods

### Antitumour Activity of EDO-S101 Against Different Xenograft Models of Human Soft Tissue Sarcoma

### EDO-S101 and Control Compounds

EDO-S101 was provided by EDO MundiPharma, and synthesised as described in Example 6 of WO-A-2010/085377.
Irinotecan was purchased from Actavis.
Doxorubicin was purchased from Sigma-Aldrich, St. Louis, MO.

Test system: Mice

Experiments were performed with female Athymic nude mice, 9-10 weeks old obtained from Harlan Laboratories (Udine, Italy). They were maintained under Specific Pathogen Free conditions with constant temperature and humidity, according to the institutional guidelines.

Mice were identified by ear tag.

Study groups and treatment regimens Study groups are listed below (at least eight mice for each group):
Vehicles
EDO-S101 80 mg/kg i.v. single dose
EDO-S101 60 mg/kg i.v. once a week per three weeks (q7dx3, one week observation time followed by an eventual subsequent cycle)
EDO-S101 40 mg/kg i.v. once a week per three weeks (q7dx3, one week observation time followed by an eventual subsequent cycle)
Irinotecan (TC-71 model) or Doxorubicin (DD013 model)

EDO-S101 was administered i.v. in a volume of 10 ml/kg. Irinotecan 50 mg/kg was administered intravenously, once a week for three weeks, in a volume of 10 ml/kg. Doxorubicin 8 mg/kg was administered intravenously, once a week for two weeks, in a volume of 10 ml/kg.

### Antitumour Activity and Drug Tolerability

Tumour growth mice was monitored two/three times a week (depending on tumour growth rate, to avoid unnecessary stress to the animals) by Vernier caliper. Mice were sacrificed when tumour reached a mean weight of 2±0.5 gr. The antitumour activity will be expressed as T/C %, where T and C were the mean tumour weight of treated and control groups, respectively. Mice were monitored daily and weighted at least two times a week. Tolerability was evaluated on the basis of body weight loss (BWL) and clinical observation.

### Statistical Analysis

Statistical analysis was performed with GraphPad Prism version 6.01 software (GraphPad software, Inc., La Jolla, CA, USA). ANOVA test was performed to evaluate if there were statistically significant differences between treated and control groups.

### Example 1

### Antitumour Activity and Drug Tolerability in TC-71 Ewing Sarcoma Model

5×10⁶ TC-71 cells were inoculated subcutaneously in the right flank of mice. The growing tumour masses were measured with the aid of a Vernier caliper, and the tumour weights (1 mm³=1 mg) were calculated by the formula: length×(width)²/2. When tumour load achieved about 100 mg, mice were randomized into the experimental groups and treatment started.

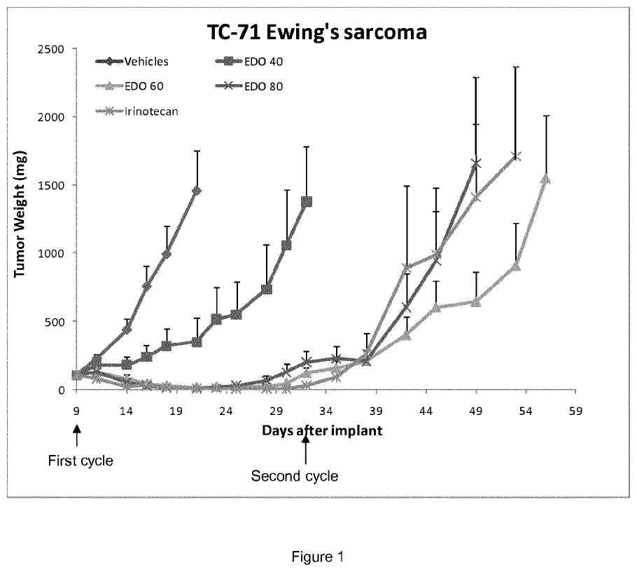

FIG. 1 shows the antitumour activity of EDO-S101 administered at different doses and schedules to TC-71 bearing mice. Both EDO-S101 administered 80 mg/kg single dose and 60 mg/kg q7dx3 were extremely effective at reducing tumour growth with optimal T/C recorded on day 21 of 0.5% and 1%, for 80 mg/kg and 60 mg/kg doses respectively.

Treatment with EDO-S101 resulted in sustained tumour regressions in all of the treated mice with complete regressions (CR) in 4 out of 8 and 3 out of 8 mice in the 80 mg/kg and 60 mg/kg groups, respectively. The administration of a second cycle of treatments at both 80 and 60 mg/kg further delayed tumour re-growth without being able to arrest it. At the end of the experiment, one mouse in the group receiving 80 mg/kg and two mice in the 60 mg/kg group were still in CR.

EDO-S101 administered at 40 mg/kg q7dx3 was effective in arresting tumour growth compared to untreated tumours with an optimal T/C of 24% (day 21). Although treatment with EDO-S101 under this dose regimen resulted in a delay in tumour growth, no tumour regression in tumour volume was observed. The group treated with 40 mg/kg EDO-S101 were not administered a second dose cycle because of the tumour dimensions (mean TW 1055.5 mg on day 30).

Irinotecan was used as positive control. Administration of 50 mg/kg of Ironotecan q7dx3 to tumour-bearing mice resulted in tumour regression in all mice with 4 mice exhibiting sustained CR up to the conclusion of the experiment. The recorded optimal T/C was 0.4% on day 21.

Tumour weights for each animal and day are reported in Table 1. The statistical analysis is reported in Table 2.

The treatments with EDO-S101 40 mg/kg were well tolerated with a maximum BWL of 5.4% recorded on day 10, 24 h after the administration of the first dose. One mouse in the group was found dead for unknown reasons, probably not ascribable to the drug since all the other mice in the treatment group did not show signs of toxicity. In the group treated with 60 mg/kg EDO-S101, a maximum BWL of 11.7% was observed on day 25, 48 h after the administration of the third dose. The second cycle appeared to be well tolerated with BWL consistently <9% for all dose regimens. At the highest dose of 80 mg/kg of EDO-S101, a maximum body weight loss of 19.3% on day 14 was observed, 5 days after administration, indicating some dose-related toxicity. Nevertheless, the mice recovered within 10 days (day 24) and the second cycle was tolerated without significant body weight loss. Irinotecan 50 mg/kg was well tolerated with a maximum BWL of 7.5% recorded on day 25. Table 3 reports the body weights recorded throughout the experiment.

EDO-S101 was shown to be effective against Ewing sarcoma. A dose of 60 mg/kg EDO-S101, administered q7dx3 resulted to be the best choice both in terms of efficacy and tolerability.

TABLE 1
TC-71 tumour weight (mg) during the in vivo evaluation of EDO-S101 (treatments started on day 9).

| Vehicles | Days from inoculum | | | | | |
|---|---|---|---|---|---|---|
| # | 9 | 11 | 14 | 16 | 18 | 21 |
| 744 | 178.9 | 341.7 | 691.8 | 1415.0 | 2070.9 | 1965.7 |
| 702 | 149.6 | 316.3 | 615.0 | 667.8 | 851.4 | 1018.5 |
| 741 | 130.8 | 300.9 | 681.7 | 1105.6 | 1325.0 | 2596.4 |
| 756 | 102.1 | 158.4 | 177.5 | 258.9 | 234.3 | 286.0 |
| 711 | 93.9 | 260.3 | 552.7 | 1177.2 | 1360.1 | 2373.9 |
| 754 | 79.1 | 201.7 | 356.4 | 608.2 | 931.6 | 1524.5 |
| 719 | 56.8 | 142.7 | 262.9 | 519.4 | 767.0 | 1411.6 |
| 705 | 32.0 | 79.2 | 157.1 | 283.4 | 380.7 | 448.3 |
| mean | 102.9 | 225.2 | 436.9 | 754.4 | 990.1 | 1453.1 |
| se | 17.2 | 33.3 | 79.3 | 151.8 | 208.4 | 298.4 |

| EDO80 | Days from inoculum | | | | | | | |
|---|---|---|---|---|---|---|---|---|
| # | 9 | 11 | 14 | 16 | 18 | 21 | 23 | 25 |
| 710 | 222.4 | 369.7 | 209.5 | 124.6 | 68.6 | 23.4 | 16.8 | 38.4 |
| 747 | 133.9 | 200.9 | 68.4 | 34.0 | 8.3 | 3.4 | 2.1 | 3.8 |
| 712 | 113.7 | 133.4 | 34.6 | 11.8 | 7.4 | 5.3 | 8.6 | 14.0 |
| 758 | 97.0 | 118.7 | 32.5 | 10.2 | 3.1 | 10.0 | 35.1 | 74.0 |
| 738 | 83.4 | 55.8 | 24.2 | 8.0 | 4.3 | 1.0 | 2.4 | CR |
| 726 | 80.5 | 65.3 | 15.6 | 5.9 | 7.1 | 2.5 | 1.2 | CR |
| 716(nn) | 57.3 | 49.8 | 11.4 | 3.2 | 2.3 | CR | CR | 4.1 |
| 727 | 45.5 | 45.0 | 17.0 | 6.8 | 4.9 | 5.4 | CR | CR |
| mean | 104.2 | 129.8 | 51.7 | 25.6 | 13.2 | 7.3 | 11.0 | 26.9 |
| se | 19.7 | 39.2 | 23.4 | 14.6 | 7.9 | 2.9 | 5.4 | 13.4 |

| EDO80 | Days from inoculum | | | | | | | |
|---|---|---|---|---|---|---|---|---|
| # | 28 | 30 | 32 | 35 | 38 | 42 | 45 | 49 |
| 710 | 117.5 | 220.0 | 340.2 | 319.1 | 239.8 | 598.5 | 790.9 | 1094.7 |
| 747 | 4.1 | 18.1 | 64.8 | 49.1 | 32.6 | 77.8 | 209.0 | 481.3 |
| 712 | 59.4 | 185.0 | 238.3 | 343.5 | 310.8 | 1030.8 | 1741.1 | 3462.7 |
| 758 | 203.9 | 415.0 | 594.3 | 692.9 | 670.0 | 1865.6 | 2763.5 | 4619.8 |
| 738 | CR | 12.8 | 37.0 | 34.3 | 56.6 | 237.5 | 370.1 | 622.1 |
| 726 | CR | CR | CR | CR | CR | CR | CR | CR |
| 716(nn) | 4.8 | 9.7 | 67.8 | 68.2 | 43.4 | 211.3 | 359.3 | 803.9 |
| 727 | 4.6 | 23.2 | 56.2 | 74.5 | 75.1 | 191.6 | 378.9 | 515.2 |
| mean | 65.7 | 126.3 | 199.8 | 225.9 | 204.0 | 601.9 | 944.7 | 1657.1 |
| se | 33.2 | 58.6 | 78.6 | 92.1 | 87.7 | 244.4 | 361.9 | 633.2 |

| EDO60 | Days from inoculum | | | | | | | | |
|---|---|---|---|---|---|---|---|---|---|
| # | 9 | 11 | 14 | 16 | 18 | 21 | 23 | 25 | 28 |
| 709 | 304.5 | 394.6 | 307.3 | 187.0 | 122.1 | 77.3 | 108.1 | 94.0 | 66.2 |
| 723 | 124.5 | 149.3 | 81.3 | 29.3 | 9.6 | 2.1 | 0.4 | 1.1 | CR |
| 749 | 100.8 | 134.2 | 71.3 | 35.4 | 29.0 | 17.0 | 31.9 | 48.5 | 28.4 |
| 740 | 87.5 | 64.9 | 29.0 | 11.5 | 6.2 | 3.3 | 5.7 | 5.7 | 13.6 |
| 721 | 82.1 | 141.3 | 51.6 | 23.9 | 12.2 | 5.5 | 0.6 | CR | CR |
| 735 | 70.7 | 60.8 | 20.5 | 10.7 | 3.4 | 3.6 | 2.1 | 9.0 | 3.3 |
| 713 | 52.7 | 70.8 | 32.0 | 8.6 | 7.9 | 3.3 | 3.3 | 3.2 | 2.9 |
| 729 | 26.2 | 26.9 | 16.1 | 7.5 | 3.4 | 0.7 | 0.6 | 1.3 | CR |
| mean | 106.1 | 130.4 | 76.1 | 39.2 | 24.2 | 14.1 | 19.1 | 23.2 | 22.9 |
| se | 30.2 | 40.9 | 34.1 | 21.4 | 14.3 | 9.2 | 13.3 | 13.4 | 11.8 |

| EDO60 | Days from inoculum | | | | | | | | |
|---|---|---|---|---|---|---|---|---|---|
| # | 30 | 32 | 35 | 38 | 42 | 45 | 49 | 53 | 56 |
| 709 | 88.0 | 195.4 | 295.9 | 345.1 | 562.9071 | 889.1 | 971.4 | 1459.1 | 2228.2 |
| 723 | CR | CR | CR | CR | CR | CR | CR | CR | CR |
| 749 | 77.1 | 161.5 | 201.8 | 307.2 | 681.301 | 725.2 | 858.8 | 1050.5 | 2084.3 |
| 740 | 41.0 | 185.8 | 224.9 | 322.5 | 589.6573 | 1089.8 | 1414.8 | 2074.7 | 2978.8 |
| 721 | CR | CR | CR | CR | CR | CR | CR | CR | CR |
| 735 | 8.9 | 37.0 | 41.1 | 44.6 | 113.6766 | 246.5 | 475.3 | 623.0 | 1546.2 |
| 713 | 3.7 | 17.2 | 11.6 | 10.0 | 35.99503 | 56.1 | 102.8 | 179.3 | 380.7 |
| 729 | CR | CR | CR | CR | CR | CR | 29.0 | 50.8 | 76.2 |
| mean | 43.7 | 119.4 | 155.1 | 205.8 | 396.7 | 601.3 | 642.0 | 906.2 | 1549.1 |
| se | 17.2 | 38.2 | 55.0 | 73.4 | 133.4 | 194.9 | 219.6 | 317.9 | 459.2 |

| EDO40 | Days from inoculum | | | | | | | | | | |
|---|---|---|---|---|---|---|---|---|---|---|---|
| # | 9 | 11 | 14 | 16 | 18 | 21 | 23 | 25 | 28 | 30 | 32 |
| 720 | 210.6 | 398.0 | 482.6 | 732.9 | 1028.4 | 1377.1 | 1901.8 | 1964.4 | 2668.1 | 3428.9 | 3598.6 |
| 739 | 147.7 | 222.8 | 244.5 | 274.3 | 437.5 | 312.3 | 377.8 | 473.5 | 420.1 | 513.3 | 650.1 |
| 701 | 112.5 | 185.7 | 150.6 | 208.5 | 227.9 | 174.8 | 329.7 | 349.6 | 515.7 | 927.1 | 1375.3 |
| (759) | | | | | | | | | | | |
| 730 | 110.5 | 145.9 | 130.2 | 127.4 | 176.0 | 194.1 | 273.0 | 257.3 | 405.6 | 763.7 | 1254.2 |
| 707 | 85.6 | 159.8 | 133.7 | 129.2 | 114.7 | 132.0 | 220.8 | 299.0 | 505.4 | 793.9 | 1384.8 |
| 745 | 72.3 | 110.1 | 93.9 | 151.1 | 225.3 | 217.6 | 458.2 | 456.3 | 570.1 | 827.6 | 1164.4 |
| 751 | 51.9 | FD | | | | | | | | | |
| 734 | 37.4 | 40.4 | 28.7 | 31.8 | 25.7 | 30.6 | 39.9 | 35.8 | 51.6 | 134.4 | 205.0 |
| mean | 103.5 | 180.4 | 180.6 | 236.4 | 319.3 | 348.4 | 514.5 | 548.0 | 733.8 | 1055.5 | 1376.1 |
| se | 19.8 | 42.4 | 56.0 | 87.5 | 127.5 | 174.5 | 236.6 | 242.4 | 328.8 | 408.2 | 405.3 |

| Irinotecan | Days from inoculum | | | | | | | |
|---|---|---|---|---|---|---|---|---|
| # | 9 | 11 | 14 | 16 | 18 | 21 | 23 | 25 |
| 746 | 316.8 | 273.1 | 87.8 | 99.6 | 44.2 | 9.6 | 13.2 | 9.9 |
| 731 | 124.6 | 90.6 | 16.6 | 41.5 | 11.3 | 2.4 | 1.7 | CR |
| 753 | 103.5 | 45.7 | 9.9 | 23.8 | 8.8 | 6.1 | 5.4 | 3.3 |
| 708 | 88.1 | 63.3 | 17.1 | 72.8 | 28.1 | 7.5 | CR | CR |
| 715 | 77.9 | 51.1 | 13.3 | 5.1 | 1.9 | CR | CR | CR |
| 737 | 61.1 | 31.1 | 9.3 | 7.0 | 2.3 | 1.9 | 1.6 | CR |
| 732 | 51.2 | 28.2 | 7.3 | 6.9 | 5.1 | 2.2 | 9.2 | 7.2 |
| 704 | 28.4 | 27.9 | 19.2 | 22.9 | 16.6 | 12.9 | 10.8 | 3.1 |
| mean | 106.4 | 76.4 | 22.6 | 35.0 | 14.8 | 6.1 | 7.0 | 5.9 |
| se | 31.9 | 29.1 | 9.4 | 12.2 | 5.2 | 1.6 | 0.6 | 1.7 |

| Irinotecan | Days from inoculum | | | | | | | | |
|---|---|---|---|---|---|---|---|---|---|
| # | 28 | 30 | 32 | 35 | 38 | 42 | 45 | 49 | 53 |
| 746 | 6.8 | 15.4 | 83.7 | 223.4 | 713.8 | 2687.8 | 2443.2 | 2887.4 | 3331.8 |
| 731 | CR | CR | CR | CR | CR | CR | CR | CR | CR |
| 753 | 1.5 | 2.7 | 8.2 | 42.7 | 105.6 | 327.2 | 627.6 | 1184.6 | 1062.6 |
| 708 | CR | CR | CR | CR | CR | CR | CR | CR | CR |
| 715 | CR | CR | CR | CR | CR | CR | CR | CR | CR |
| 737 | CR | CR | CR | CR | CR | CR | CR | CR | CR |
| 732 | 3.3 | 3.8 | 12.1 | 36.0 | 74.5 | 346.6 | 589.7 | 1244.3 | 2135.1 |
| 704 | 2.5 | 2.6 | 9.2 | 53.8 | 145.5 | 199.0 | 305.0 | 316.1 | 308.0 |
| mean | 3.5 | 6.1 | 28.3 | 88.9 | 259.8 | 890.2 | 991.4 | 1408.1 | 1709.4 |
| se | 1.2 | 3.1 | 18.5 | 45.0 | 152.0 | 600.1 | 489.3 | 536.8 | 658.0 |

FD: Found Dead
CR: Complete Response

TABLE 2
Antitumour activity of EDO-S101 against TC-71
Ewing's sarcoma xenograft: statistical analysis

| | Days from inoculum | | | |
|---|---|---|---|---|
| | 14 | 16 | 18 | 21 |
| Vehicles vs. | p < 0.05 | p < 0.0001 | p < 0.0001 | p < 0.0001 |
| EDO 80 | | | | |
| Vehicles vs. | p < 0.05 | p < 0.0001 | p < 0.0001 | p < 0.0001 |
| EDO60 | | | | |
| Vehicles vs. | ns | p < 0.001 | p < 0.0001 | p < 0.0001 |
| EDO40 | | | | |
| Vehicles vs. | p < 0.01 | p < 0.0001 | p < 0.0001 | p < 0.0001 |
| Irinotecan | | | | |

TABLE 3
Mouse body weight (g) during the in vivo evaluation of EDO-S101 in TC-71 Ewing's sarcoma
xenograft (treatments started on day 9).

| Vehicles | Days from tumour inoculum | | | | | | |
|---|---|---|---|---|---|---|---|
| # | 9 | 10 | 11 | 14 | 16 | 18 | 21 |
| 744 | 28.3 | 27 | 28.3 | 28.9 | 29.7 | 30.2 | 25.8 |
| 702 | 24.3 | 23.5 | 24.6 | 25.2 | 25.7 | 24.6 | 24.8 |
| 741 | 29.8 | 28.8 | 30.1 | 30.8 | 31.5 | 31.4 | 32 |
| 756 | 28.3 | 27.5 | 27.9 | 28.2 | 28.4 | 28.4 | 27.6 |
| 711 | 25.3 | 25.9 | 26.6 | 27.7 | 28.2 | 28.7 | 28.2 |
| 754 | 27.9 | 26.8 | 27.4 | 27.7 | 28.8 | 29.8 | 29.9 |
| 719 | 28.3 | 27.7 | 28.7 | 29.2 | 30 | 30.1 | 30 |
| 705 | 28.1 | 26.9 | 27.6 | 27.9 | 25.7 | 28.8 | 29 |
| Mean | 27.5 | 26.8 | 27.7 | 28.2 | 28.5 | 29.0 | 28.4 |
| se | 0.6 | 0.6 | 0.6 | 0.6 | 0.7 | 0.7 | 0.8 |
| BWL % | 0.0 | −2.8 | 0.4 | 2.4 | 3.5 | 5.3 | 3.2 |

| EDO80 | Days from tumour inoculum | | | | | | | |
|---|---|---|---|---|---|---|---|---|
| # | 9 | 10 | 11 | 14 | 16 | 18 | 21 | 23 |
| 710 | 28.4 | 25.1 | 24.9 | 21.8 | 23.3 | 24.1 | 25.7 | 26.1 |
| 747 | 25.3 | 22.8 | 22.6 | 19.4 | 19.7 | 22.2 | 22.6 | 23.8 |
| 712 | 26.1 | 24 | 23.5 | 22.4 | 24.2 | 24.8 | 26.5 | 26.6 |
| 758 | 26.6 | 24.8 | 24.7 | 23.5 | 25.1 | 26 | 26.2 | 26.6 |
| 738 | 28.5 | 26.8 | 26 | 23.2 | 24.8 | 27 | 27.4 | 27.9 |
| 726 | 27.5 | 24.4 | 24.4 | 23.2 | 24.4 | 26.1 | 26.3 | 26.6 |
| 716(nn) | 32 | 28.6 | 27.9 | 23.8 | 26.9 | 28 | 29.6 | 30.1 |
| 727 | 28.7 | 25.5 | 24.3 | 22.7 | 22.9 | 23.5 5 | 25.2 | 25.2 |
| Mean | 27.9 | 25.3 | 24.8 | 22.5 | 23.9 | 25.2 | 26.2 | 26.6 |
| se | 0.7 | 0.6 | 0.6 | 0.5 | 0.7 | 0.7 | 0.7 | 0.7 |
| BWL % | 0.0 | −9.5 | −11.1 | −19.3 | −14.3 | −9.6 | −6.1 | −4.6 |

| EDO80 | Days from tumour inoculum | | | | | | | | |
|---|---|---|---|---|---|---|---|---|---|
| # | 25 | 28 | 30 | 32 | 35 | 38 | 42 | 46 | 49 |
| 710 | 27 | 27.9 | 28.2 | 27.4 | 24.4 | 24.4 | 26.3 | 28.6 | 29.8 |
| 747 | 25.1 | 25.4 | 25.3 | 24.9 | 21.9 | 21.8 | 23.1 | 25.3 | 26 |
| 712 | 27.9 | 28.7 | 27.3 | 26.6 | 24.8 | 25.1 | 27 | 28.3 | 30 |
| 758 | 27.7 | 28.1 | 27.9 | 27.4 | 26.1 | 25.3 | 28 | 30 | 32.3 |
| 738 | 28.8 | 30.9 | 29.7 | 29 | 26.6 | 26 | 28.2 | 29.2 | 31 |
| 726 | 27 | 28.5 | 27.9 | 27.4 | 24.8 | 25 | 26.6 | 28 | 28.3 |
| 716(nn) | 31.4 | 32.6 | 32.2 | 31.8 | 27.4 | 27.6 | 30.9 | 32.5 | 34.1 |
| 727 | 27.2 | 29 | 29.2 | 29.4 | 25.1 | 24.8 | 26.5 | 28.2 | 29.9 |
| Mean | 27.8 | 28.9 | 28.5 | 28.0 | 25.1 | 25.0 | 27.1 | 28.8 | 30.2 |
| se | 0.6 | 0.8 | 0.7 | 0.7 | 0.6 | 0.6 | 0.8 | 0.7 | 0.9 |
| BWL % | −0.4 | 3.6 | 2.1 | 0.4 | −9.9 | −10.4 | −2.9 | 3.1 | 8.2 |
| EDO60 | Days from tumour inoculum | | | | | | | | |
| # | 9 | 10 | 11 | 14 | 16 | 18 | 21 | 23 | 25 |
| 709 | 26.7 | 24.4 | 24.4 | 24 | 24.8 | 24.8 | 24.6 | 25 | 24.1 |
| 723 | 25.4 | 25.3 | 25.1 | 24.4 | 25.4 | 23.5 | 20.9 | 21.2 | 20.9 |
| 749 | 27.9 | 25.5 | 25.3 | 25 | 25.9 | 24.4 | 24.7 | 25.1 | 23.5 |
| 740 | 27.6 | 25.7 | 25.5 | 25.8 | 27 | 26.2 | 26 | 26.6 | 25.7 |
| 721 | 29.2 | 27.2 | 26.6 | 27.4 | 27.4 | 26.1 | 26 | 25.7 | 25.3 |
| 735 | 29.2 | 28.1 | 27.2 | 27.7 | 28.7 | 27.4 | 27.9 | 27.8 | 26.6 |
| 713 | 26.5 | 24.1 | 23.6 | 24.5 | 25 | 24.8 | 25 | 24.8 | 25.7 |
| 729 | 28.1 | 24.8 | 24 | 24 | 25.2 | 24 | 23.9 | 24 | 23.1 |
| Mean | 27.6 | 25.6 | 25.2 | 25.4 | 26.2 | 25.2 | 24.9 | 25.0 | 24.4 |
| se | 0.5 | 0.5 | 0.4 | 0.5 | 0.5 | 0.5 | 0.7 | 0.7 | 0.7 |
| BWL % | 0.0 | −7.0 | −8.6 | −8.1 | −5.1 | −8.8 | −9.8 | −9.2 | −11.7 |

| EDO60 | Days from tumour inoculum | | | | | | | | | |
|---|---|---|---|---|---|---|---|---|---|---|
| # | 28 | 30 | 32 | 35 | 38 | 42 | 46 | 49 | 53 | 56 |
| 709 | 25 | 24.8 | 26.3 | 25.8 | 25.8 | 25.5 | 25.1 | 25.7 | 27 | 29.2 |
| 723 | 21.5 | 22.2 | 21.9 | 21 | 22.8 | 21.8 | 20.7 | 21.2 | 22.9 | 23.8 |
| 749 | 25 | 25.3 | 26.8 | 25.9 | 24.8 | 25.7 | 25.5 | 25.7 | 26.6 | 28 |
| 740 | 26.8 | 27.4 | 27.4 | 25.7 | 27.4 | 27.5 | 26.9 | 27 | 29.6 | 30 |
| 721 | 26.2 | 27 | 28.5 | 27.5 | 27.3 | 25.8 | 24.8 | 26.1 | 27.1 | 29.1 |
| 735 | 27.8 | 28.3 | 28.7 | 27.4 | 27.8 | 28.3 | 27.3 | 27 | 28.1 | 30 |
| 713 | 25.6 | 25.6 | 26.5 | 25.8 | 25.8 | 26 | 25.7 | 25.7 | 26.3 | 27.9 |
| 729 | 24.3 | 24 | 25.3 | 23.8 | 24.4 | 25.7 | 25 | 24.4 | 26.1 | 27 |
| Mean | 25.3 | 25.6 | 26.4 | 25.4 | 25.8 | 25.8 | 25.1 | 25.4 | 26.7 | 28.1 |
| se | 0.7 | 0.7 | 0.8 | 0.7 | 0.6 | 0.7 | 0.7 | 0.7 | 0.7 | 0.7 |
| BWL% | −8.3 | −7.3 | −4.2 | −8.0 | −6.6 | −6.5 | −8.9 | −8.1 | −3.1 | 2.0 |

| EDO40 | Days from tumour inoculum | | | | | | | | | | | |
|---|---|---|---|---|---|---|---|---|---|---|---|---|
| # | 9 | 10 | 11 | 14 | 16 | 18 | 21 | 23 | 25 | 28 | 30 | 32 |
| 720 | 29.7 | 28.7 | 29.3 | 29.6 | 31.5 | 30 | 30.4 | 31.7 | 30.9 | 33.8 | 35 | 36 |
| 739 | 26.4 | 25.4 | 25.7 | 26 | 26.3 | 25.3 | 24.7 | 25.3 | 24.2 | 25.5 | 25.7 | 26.8 |
| 701 (759) | 26.2 | 24.9 | 25.2 | 25.5 | 26.2 | 24.8 | 25.4 | 26.2 | 25.2 | 27 | 27.3 | 27.3 |
| 730 | 27.8 | 25.7 | 26.2 | 27 | 26.8 | 26.2 | 26.1 | 26.8 | 26.1 | 27 | 27.6 | 28.1 |
| 707 | 27.7 | 25.8 | 25.4 | 26.4 | 28.1 | 25.8 | 26.8 | 27.9 | 27.3 | 28.6 | 29.6 | 28.7 |
| 745 | 24.8 | 22.9 | 23.6 | 24.8 | 24.8 | 24.3 | 24 | 25 | 24.4 | 25.5 | 26.6 | 26.5 |
| 751 | 28.3 | FD | | | | | | | | | | |
| 734 | 25.4 | 25.7 | 26.2 | 26.9 | 26.8 | 26.5 | 26.1 | 27 | 25.7 | 26.3 | 27.4 | 27.2 |
| Mean | 27.0 | 25.6 | 25.9 | 26.6 | 27.2 | 26.1 | 26.2 | 27.1 | 26.3 | 27.7 | 28.5 | 28.7 |
| se | 0.6 | 0.6 | 0.6 | 0.5 | 0.8 | 0.7 | 0.7 | 0.8 | 0.8 | 1.0 | 1.1 | 1.2 |
| BWL % | 0.0 | −5.4 | −4.0 | −1.6 | 0.7 | −3.4 | −3.0 | 0.3 | −2.9 | 2.3 | 5.3 | 6.0 |

| Irinotecan | Days from tumour inoculum | | | | | | | | |
|---|---|---|---|---|---|---|---|---|---|
| # | 9 | 10 | 11 | 14 | 16 | 18 | 21 | 23 | 25 |
| 746 | 27.7 | 25.3 | 25.7 | 25.3 | 26.2 | 25.7 | 26.7 | 26.2 | 25.3 |
| 731 | 29.4 | 26.5 | 26.9 | 27.3 | 28.1 | 26.6 | 27.4 | 27 | 26.3 |
| 753 | 28.6 | 26.7 | 26.7 | 28 | 27.7 | 27.1 | 27.1 | 27 | 25.7 |
| 708 | 25.7 | 24 | 23.5 | 25.5 | 25.5 | 24.9 | 24.5 | 24.7 | 24.4 |
| 715 | 29.4 | 26.6 | 26 | 27.9 | 27.9 | 26.6 | 27 | 27.1 | 25.7 |
| 737 | 26.1 | 24.9 | 25.3 | 26.2 | 25.7 | 25.5 | 26 | 26.2 | 25 |
| 732 | 26 | 26 | 26.1 | 26.6 | 26.3 | 25.8 | 26 | 26.6 | 25.8 |
| 704 | 26.8 | 25.6 | 24.8 | 26.9 | 27.5 | 25.4 | 25.7 | 26.6 | 24.7 |
| Mean | 27.4 | 25.8 | 25.6 | 26.9 | 27.0 | 26.0 | 26.2 | 26.5 | 25.4 |
| se | 0.6 | 0.4 | 0.4 | 0.3 | 0.4 | 0.3 | 0.4 | 0.3 | 0.2 |
| BWL % | 0.0 | −6.1 | −6.6 | −1.9 | −1.7 | −5.3 | −4.3 | −3.5 | −7.5 |
| Irinotecan | Days from tumour inoculum | | | | | | | | |
| # | 28 | 30 | 32 | 35 | 38 | 42 | 46 | 49 | 53 |
| 746 | 27.2 | 27 | 27 | 28.3 | 27.3 | 29.4 | 28.7 | 30.7 | 31.8 |
| 731 | 28.1 | 28.5 | 28.5 | 30.2 | 28.9 | 30.9 | 29.4 | 30.5 | 29.5 |
| 753 | 26.1 | 27 | 27.4 | 27.3 | 26.1 | 27.4 | 27.9 | 28.5 | 25 |
| 708 | 24.2 | 25.3 | 25.2 | 25.9 | 26.1 | 26.1 | 26.1 | 25.5 | 25.8 |
| 715 | 27 | 27.4 | 27.4 | 28.7 | 26.8 | 28.6 | 27.8 | 28.3 | 27.4 |
| 737 | 26.6 | 27 | 26.6 | 27.1 | 26.6 | 28 | 27.4 | 27.9 | 29 |
| 732 | 26.9 | 27 | 26.3 | 27.9 | 28.1 | 28.6 | 28 | 29.2 | 29.8 |
| 704 | 25.6 | 27.6 | 27.6 | 28.2 | 28 | 29.1 | 28.2 | 29.1 | 29.9 |
| Mean | 26.4 | 27.1 | 27.0 | 27.9 | 27.2 | 28.4 | 27.8 | 28.4 | 28.1 |
| se | 0.4 | 0.3 | 0.4 | 0.5 | 0.4 | 0.5 | 0.3 | 0.5 | 0.7 |
| BWL % | −3.9 | −1.1 | −1.6 | 1.7 | −0.7 | 3.5 | 1.5 | 3.6 | 2.3 |

FD: Found Dead
CR: Complete Response

### Example 2

### Antitumour Activity and Drug Tolerability in DD013 Dedifferentiated Sarcoma Model

DD013 dedifferentiated liposarcoma were established and maintained in nude mice as described in Frapolli R. et al Clin Cancer Res 2010 16: 4958-4967. Briefly, xenografts were obtained by transplanting 3-4 mm tumour fragments s.c. in the flanks of mice. The growing tumour masses were measured with the aid of a Vernier caliper, and the tumour weights (TW, 1 mm³=1 mg) were calculated by the formula: length×(width) 2/2. When tumour load achieved about 200-400 mg, mice were randomized into the experimental groups.

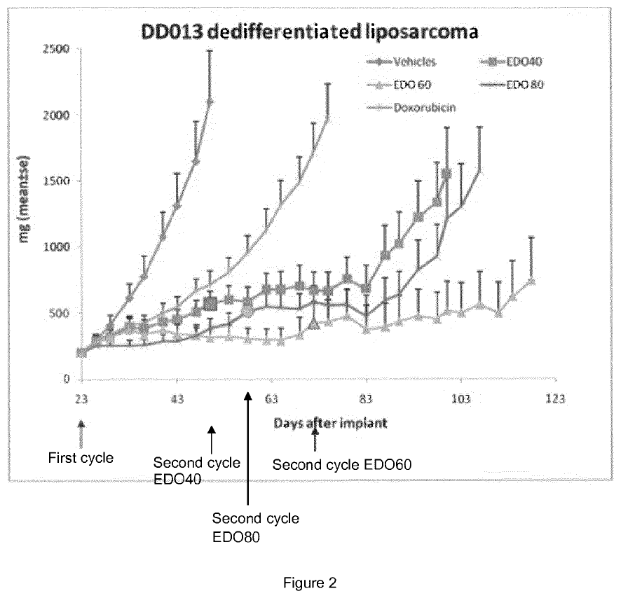

FIG. 2 shows the antitumour activity of EDO-S101 administered at different doses and schedules to DD013 bearing mice. The second cycles were administered when tumours started to regrow on day 50, 58 and 72 for EDO-S101 doses of 40, 80 and 60 mg/kg, respectively. Treatment of tumour-bearing mice with EDO-S101 (80 and 60 mg/kg) induced tumour growth arrest with an optimal T/C on day 50 of 18% and 14%, for 80 and 60 mg/kg respectively. Despite similar T/C values for these dose regimens, administration of 60 mg/kg q7dx3 resulted in a more sustained arrest of tumour growth, compared to administration of a single dose of 80 mg/kg. The administration of a second cycle of EDO-S101 (60 mg/kg) sustained tumour growth arrest, and significantly reduced tumour growth over a prolonged period towards the conclusion of the experiment. Tumour growth arrest was also observed following the administration of a second cycle in the group receiving 80 mg/kg of EDO-S101.

Mice treated with EDO-S101 (40 mg/kg) q7dx3 revealed reduced tumour growth, but without a complete arrest. Administration of the second cycle of treatment, however, promoted tumour growth arrest under this dose regimen. The optimal T/C was 27.2% on day 50.

Doxorubicin was used as positive control. Administration of 8 mg/kg of doxorubicin q7dx2 resulted in reduced tumour growth rate compared to the negative control, with an optimal T/C of 34.1%. No arrest of tumour growth was observed in mice treated with doxorubicin. Tumour weights for each animal and day are reported in table 4. The statistical analysis is reported in table 5.

BWL<5% was recorded at all doses and schedules. One mouse in the group treated with 80 mg/kg EDO-S101 was sacrificed due to a rapid BWL>30% occurring between day 75 and 79 from inoculum (about three weeks after the administration of the second cycle) of treatments.

Necropsy revealed no macroscopically detectable causes of death. Doxorubicin 8 mg/kg was well tolerated with a maximum BWL of 5.2% recorded on day 33. Table 6 reports the mouse body weights recorded throughout the experiment.

TABLE 4
DD0113 tumour weight (mg) during the in vivo evaluation of EDO-S101
(treatments started on day 23).

| Vehicles | Days from inoculum | | | | | | | | |
|---|---|---|---|---|---|---|---|---|---|
| # | 23 | 26 | 29 | 33 | 36 | 40 | 43 | 47 | 50 |
| 5 | 389.8 | 534.0 | 830.6 | 1138.4 | 1630.3 | 2043.6 | 2584.8 | 3155.1 | 3991.9 |
| 20 | 272.3 | 420.7 | 600.3 | 839.1 | 1043.5 | 1436.5 | 1723.4 | 2182.9 | 2403.8 |
| 34 | 246.5 | 353.8 | 551.5 | 670.0 | 931.2 | 1148.0 | 1417.8 | 1682.9 | 2114.3 |
| 69 | 224.6 | 208.9 | 272.4 | 458.5 | 524.2 | 775.6 | 948.2 | 1139.0 | 1593.6 |
| 17 | 187.0 | 358.8 | 471.1 | 813.2 | 1023.0 | 1489.2 | 1702.9 | 2394.3 | 3198.3 |
| 11 | 159.0 | 286.4 | 401.9 | 784.3 | 924.7 | 1346.0 | 1843.4 | 2206.4 | 2947.1 |
| 16 | 118.3 | 139.0 | 159.3 | 251.3 | 259.6 | 304.2 | 302.1 | 372.1 | 395.4 |
| 29 | 102.5 | 174.7 | 197.2 | 324.8 | 353.9 | 606.3 | 630.8 | 746.7 | 972.6 |
| 10 | 93.2 | 149.7 | 169.3 | 235.0 | 306.9 | 543.2 | 649.3 | 955.2 | 1279.3 |
| Mean | 199.2 | 291.8 | 406.0 | 612.7 | 777.4 | 1077.0 | 1311.4 | 1648.3 | 2099.6 |
| se | 31.9 | 45.3 | 76.5 | 104.1 | 150.8 | 186.5 | 244.1 | 302.9 | 385.5 |

| EDO40 | Days from inoculum | | | | | | | |
|---|---|---|---|---|---|---|---|---|
| # | 23 | 26 | 29 | 33 | 36 | 40 | 43 | 47 |
| 3 | 376.6 | 541.5 | 577.2 | 733.4 | 740.0 | 853.1 | 874.8 | 951.5 |
| 49 | 272.5 | 407.4 | 528.9 | 620.3 | 636.3 | 647.3 | 620.0 | 867.8 |
| 46 | 234.4 | 341.7 | 304.2 | 386.0 | 313.7 | 394.8 | 382.2 | 374.0 |
| 43 | 224.8 | 312.6 | 326.8 | 398.8 | 375.3 | 445.1 | 452.6 | 447.7 |
| 36 | 191.1 | 284.8 | 350.2 | 400.6 | 376.9 | 470.3 | 614.9 | 502.6 |
| 35 | 172.9 | 244.9 | 315.8 | 410.8 | 408.8 | 402.2 | 419.5 | 504.6 |
| 44 | 124.8 | 208.4 | 218.0 | 307.5 | 294.7 | 363.7 | 359.8 | 551.2 |
| 58 | 103.7 | 138.3 | 150.5 | 165.2 | 125.0 | 150.4 | 143.6 | 155.1 |
| 54 | 86.8 | 72.8 | 101.4 | 127.5 | 151.8 | 174.0 | 194.8 | 211.9 |
| Mean | 198.6 | 283.6 | 319.2 | 394.4 | 380.3 | 433.4 | 451.3 | 507.4 |
| se | 30.5 | 47.0 | 52.4 | 64.3 | 67.2 | 72.5 | 75.4 | 88.3 |
| EDO40 | Days from inoculum | | | | | | | |
| # | 50 | 54 | 58 | 62 | 65 | 69 | 72 | 75 |
| 3 | 1015.8 | 1218.3 | 1198.4 | 1270.3 | 1227.3 | 1303.1 | 1437.5 | 1469.1 |
| 49 | 910.3 | 849.1 | 939.5 | 1057.1 | 1277.6 | 1503.3 | 1078.7 | 1076.6 |
| 46 | 477.7 | 407.8 | 361.1 | 400.9 | 371.1 | 405.2 | 409.0 | 425.4 |
| 43 | 675.3 | 650.3 | 647.5 | 778.6 | 690.9 | 866.7 | 909.6 | 836.1 |
| 36 | 556.8 | 641.5 | 526.9 | 706.2 | 485.1 | 447.9 | 438.2 | 429.0 |
| 35 | 593.9 | 566.8 | 517.8 | 694.4 | 740.8 | 712.8 | 702.4 | 742.0 |
| 44 | 509.8 | 653.1 | 678.7 | 795.3 | 906.7 | 742.0 | 638.6 | 670.3 |
| 58 | 166.1 | 138.4 | 148.4 | 127.1 | 153.2 | 124.4 | 146.3 | 117.4 |
| 54 | 228.8 | 270.3 | 238.9 | 271.1 | 247.5 | 223.2 | 267.4 | 229.4 |
| Mean | 570.5 | 599.5 | 584.1 | 677.9 | 677.8 | 703.2 | 669.8 | 666.1 |
| se | 92.6 | 106.1 | 110.7 | 121.6 | 134.9 | 155.8 | 138.1 | 142.1 |

| EDO40 | Days from inoculum | | | | | | |
|---|---|---|---|---|---|---|---|
| # | 79 | 83 | 87 | 90 | 94 | 98 | 100 |
| 3 | 1640.1 | 1758.9 | 2103.7 | 2135.1 | 2516.4 | 2716.0 | 3162.4 |
| 49 | 1320.3 | 1083.6 | 1649.4 | 1918.5 | 2036.1 | 1993.4 | 2274.9 |
| 46 | 560.8 | 394.8 | 526.5 | 598.6 | 820.3 | 913.4 | 963.9 |
| 43 | 876.4 | 1023.8 | 1294.1 | 1450.6 | 1686.1 | 1775.6 | 2276.4 |
| 36 | 387.3 | 391.4 | 457.1 | 463.8 | 542.4 | 446.5 | 533.5 |
| 35 | 854.9 | 677.5 | 994.3 | 1104.6 | 1322.5 | 1827.2 | 2007.9 |
| 44 | 755.9 | 567.8 | 1055.8 | 1165.7 | 1662.8 | 1853.1 | 2178.4 |
| 58 | 144.0 | 83.9 | 95.8 | 119.3 | 133.6 | 145.4 | 160.4 |
| 54 | 271.2 | 173.5 | 245.9 | 249.5 | 318.3 | 371.6 | 419.7 |
| Mean | 756.8 | 683.9 | 935.9 | 1022.8 | 1226.5 | 1338.0 | 1553.1 |
| se | 162.8 | 176.2 | 223.5 | 240.3 | 273.4 | 297.1 | 350.5 |

| EDO60 | Days from inoculum | | | | | | | | | |
|---|---|---|---|---|---|---|---|---|---|---|
| # | 23 | 26 | 29 | 33 | 36 | 40 | 43 | 47 | 50 | 54 |
| 39 | 390.7 | 581.5 | 713.8 | 959.2 | 752.3 | 858.2 | 788.1 | 784.4 | 753.8 | 868.2 |
| 23 | 270.8 | 439.5 | 542.7 | 709.9 | 694.9 | 638.0 | 594.1 | 617.7 | 533.9 | 564.0 |
| 7 | 256.0 | 349.4 | 408.5 | 434.0 | 374.9 | 442.3 | 396.4 | 372.6 | 327.6 | 225.0 |
| 40 | 208.0 | 277.6 | 245.2 | 230.8 | 195.0 | 178.1 | 196.5 | 162.6 | 176.0 | 134.2 |
| 53 | 186.9 | 282.9 | 337.8 | 388.5 | 320.6 | 462.9 | 359.1 | 384.0 | 334.2 | 413.0 |
| 42 | 149.8 | 149.7 | 127.5 | 156.7 | 151.2 | 195.4 | 165.1 | 150.5 | 138.1 | 123.8 |
| 68 | 137.4 | 162.1 | 180.6 | 179.5 | 215.2 | 211.0 | 214.6 | 209.0 | 199.7 | 227.1 |
| 52 | 98.0 | 93.0 | 129.5 | 185.1 | 208.1 | 215.3 | 247.2 | 194.4 | 257.7 | 253.8 |
| 51 | 95.4 | 117.1 | 107.7 | 114.7 | 95.8 | 139.7 | 94.4 | 96.9 | 77.1 | 80.6 |
| Mean | 199.2 | 272.5 | 310.4 | 373.1 | 334.2 | 371.2 | 339.5 | 330.2 | 310.9 | 321.1 |
| se | 31.7 | 54.4 | 70.3 | 96.3 | 78.7 | 82.8 | 75.0 | 78.3 | 71.2 | 85.1 |

| EDO60 | Days from inoculum | | | | | | | | |
|---|---|---|---|---|---|---|---|---|---|
| # | 58 | 62 | 65 | 69 | 72 | 75 | 79 | 83 | 87 |
| 39 | 862.4 | 887.6 | 998.1 | 1344.4 | 1654.5 | 1698.0 | 1977.8 | 1774.9 | 1753.1 |
| 23 | 420.0 | 395.4 | 352.1 | 347.7 | 414.7 | 457.6 | 571.8 | 335.3 | 396.9 |
| 7 | 265.4 | 217.1 | 125.3 | 142.7 | 188.6 | 170.8 | 172.6 | 86.5 | 125.1 |
| 40 | 150.8 | 153.4 | 133.1 | 96.1 | 130.6 | 110.6 | 132.6 | 56.8 | 78.1 |
| 53 | 404.6 | 418.0 | 350.2 | 371.1 | 509.4 | 516.6 | 555.7 | 415.0 | 442.6 |
| 42 | 134.5 | 111.0 | 133.4 | 86.2 | 157.5 | 123.5 | 152.1 | 71.0 | 82.4 |
| 68 | 187.9 | 183.0 | 187.4 | 191.3 | 268.5 | 307.7 | 256.1 | 193.9 | 209.1 |
| 52 | 247.8 | 255.2 | 278.4 | 367.8 | 403.5 | 441.0 | 404.7 | 426.4 | 423.2 |
| 51 | 56.8 | 63.9 | 66.8 | 63.8 | 86.1 | 70.2 | 58.1 | 32.7 | 31.4 |
| Mean | 303.3 | 298.3 | 291.6 | 334.6 | 423.7 | 432.9 | 475.7 | 376.9 | 393.6 |
| se | 80.5 | 83.6 | 94.7 | 132.9 | 161.3 | 167.6 | 197.7 | 182.3 | 178.2 |
| EDO60 | Days from inoculum | | | | | | | | |
| # | 90 | 94 | 98 | 100 | 103 | 107 | 111 | 114 | 118 |
| 39 | 1903.1 | 1967.5 | 1939.1 | 2099.8 | 2142.4 | 2405.7 | 2171.7 | 2520.5 | 3030.9 |
| 23 | 365.0 | 403.0 | 387.0 | 463.0 | 430.4 | 428.9 | 368.4 | 487.7 | 526.7 |
| 7 | 118.4 | 135.6 | 113.6 | 122.1 | 106.7 | 166.5 | 90.0 | 131.7 | 157.8 |
| 40 | 56.3 | 73.5 | 57.9 | 86.5 | 70.5 | 65.8 | 45.6 | 81.4 | 79.8 |
| 53 | 626.5 | 693.0 | 655.0 | 751.1 | 747.9 | 824.2 | 813.1 | 999.4 | 1342.5 |
| 42 | 82.7 | 95.1 | 70.1 | 87.0 | 89.6 | 103.8 | 62.2 | 119.1 | 134.3 |
| 68 | 234.7 | 279.8 | 225.1 | 297.7 | 231.0 | 255.2 | 245.9 | 284.3 | 364.6 |
| 52 | 492.6 | 598.8 | 584.9 | 718.0 | 670.9 | 777.9 | 686.9 | 935.9 | 1024.5 |
| 51 | 49.6 | 43.4 | 30.7 | 40.7 | 37.5 | 33.0 | 33.9 | 61.0 | 64.1 |
| Mean | 436.6 | 476.6 | 451.5 | 518.4 | 503.0 | 562.3 | 502.0 | 624.6 | 747.2 |
| se | 195.7 | 202.1 | 201.4 | 217.4 | 223.2 | 250.5 | 229.5 | 265.6 | 322.2 |
| EDO80 | Days from inoculum | | | | | | | | |
| # | 23 | 26 | 29 | 33 | 36 | 40 | 43 | 47 | 50 |
| 33 | 361.8 | 410.2 | 399.5 | 456.2 | 466.8 | 569.1 | 500.6 | 626.2 | 686.0 |
| 56 | 272.9 | 433.7 | 447.9 | 408.9 | 431.6 | 476.7 | 547.3 | 643.5 | 716.7 |
| 32 | 241.0 | 275.2 | 310.3 | 293.6 | 284.2 | 364.0 | 355.5 | 338.2 | 447.7 |
| 60 | 225.2 | 338.5 | 335.3 | 271.4 | 280.3 | 279.7 | 255.8 | 321.9 | 447.8 |
| 59 | 191.6 | 196.8 | 179.1 | 220.9 | 194.9 | 216.2 | 227.9 | 275.0 | 297.1 |
| 14 | 176.4 | 236.0 | 229.9 | 247.0 | 242.1 | 257.3 | 284.6 | 335.7 | 416.1 |
| 50 | 118.7 | 145.4 | 182.9 | 151.7 | 168.5 | 140.9 | 164.8 | 167.6 | 184.9 |
| 37 | 113.3 | 152.8 | 95.7 | 103.9 | 129.4 | 130.0 | 116.9 | 97.1 | 91.6 |
| 38 | 81.5 | 89.0 | 80.4 | 105.2 | 109.8 | 119.3 | 109.5 | 132.0 | 171.9 |
| Mean | 198.0 | 253.1 | 251.2 | 251.0 | 256.4 | 283.7 | 284.8 | 326.3 | 384.4 |
| se | 29.5 | 40.3 | 43.3 | 41.3 | 41.8 | 52.9 | 52.4 | 65.5 | 73.5 |

| EDO80 | Days from inoculum | | | | | | | |
|---|---|---|---|---|---|---|---|---|
| # | 54 | 58 | 62 | 65 | 69 | 72 | 75 | 79 |
| 33 | 708.8 | 920.2 | 943.1 | 1025.7 | 1144.1 | 1339.2 | 1250.1 | 1330.0 |
| 56 | 836.1 | 863.6 | 970.6 | 939.0 | 909.3 | 893.1 | 779.7 | 618.2 |
| 32 | 469.2 | 593.4 | 633.0 | 636.4 | 591.5 | 629.6 | 675.2 | 650.1 |
| 60 | 456.3 | 628.6 | 680.2 | 670.6 | 550.9 | 619.0 | 634.0 | 690.9 |
| 59 | 339.2 | 525.3 | 527.0 | 491.0 | 478.8 | 542.0 | 567.6 | 573.1 |
| 14 | 434.6 | 442.9 | 583.0 | 567.8 | 587.8 | 621.4 | 519.9 | 621.4 |
| 50 | 213.4 | 248.6 | 199.9 | 201.7 | 164.5 | 206.7 | 185.4 | 212.9 |
| 37 | 125.8 | 167.1 | 187.7 | 107.7 | 146.5 | 134.1 | 158.8 | 135.0 |
| 38 | 186.9 | 212.9 | 219.1 | 222.7 | 211.1 | 248.6 | 277.5 | 245.2 |
| Mean | 418.9 | 511.4 | 549.2 | 540.3 | 531.6 | 581.5 | 560.9 | 564.1 |
| se | 79.1 | 90.8 | 100.1 | 107.1 | 112.6 | 125.3 | 113.4 | 119.3 |
| EDO80 | Days from inoculum | | | | | | | |
| # | 83 | 87 | 90 | 94 | 98 | 100 | 103 | 107 |
| 33 | 1450.9 | 1616.4 | 1617.7 | 2114.0 | 2225.7 | 3065.0 | 2981.4 | 3037.4 |
| 56 | | | | | | | | |
| 32 | 575.5 | 647.4 | 735.1 | 1048.7 | 1385.7 | 1722.4 | 1979.3 | 2529.3 |
| 60 | 509.7 | 823.3 | 987.9 | 1242.6 | 1194.7 | 1733.1 | 1845.5 | 2157.8 |
| 59 | 400.2 | 496.0 | 466.5 | 555.8 | 782.4 | 996.2 | 1231.1 | 1395.6 |
| 14 | 507.9 | 665.8 | 740.1 | 814.4 | 918.1 | 1052.7 | 1125.6 | 1619.3 |
| 50 | 115.7 | 136.4 | 153.4 | 240.1 | 164.4 | 303.2 | 320.3 | 476.4 |
| 37 | 117.9 | 195.7 | 227.0 | 306.5 | 464.1 | 413.3 | 529.9 | 745.4 |
| 38 | 122.5 | 171.6 | 182.9 | 285.8 | 282.5 | 392.7 | 396.4 | 619.0 |
| Mean | 475.0 | 594.1 | 638.8 | 826.0 | 927.2 | 1209.8 | 1301.2 | 1572.5 |
| se | 155.3 | 172.3 | 176.4 | 226.0 | 239.1 | 332.0 | 327.1 | 333.6 |
| Doxorubicin | Days from inoculum | | | | | | | |
| # | 23 | 26 | 29 | 33 | 36 | 40 | 43 | 47 |
| 25 | 302.2 | 464.9 | 492.1 | 566.5 | 549.6 | 578.4 | 529.7 | 623.2 |
| 57 | 277.0 | 410.3 | 380.4 | 526.0 | 572.5 | 652.6 | 810.5 | 800.6 |
| 65 | 253.6 | 365.9 | 478.9 | 691.7 | 610.4 | 762.1 | 868.0 | 1056.8 |
| 61 | 228.6 | 384.0 | 477.1 | 549.0 | 519.4 | 750.9 | 780.2 | 1025.8 |
| 12 | 196.0 | 308.8 | 398.0 | 444.0 | 503.4 | 531.1 | 575.9 | 669.1 |
| 21 | 178.0 | 283.4 | 313.2 | 344.6 | 361.9 | 424.1 | 420.0 | 636.6 |
| 62 | 147.0 | 232.9 | 252.9 | 295.0 | 345.6 | 401.6 | 428.4 | 529.5 |
| 8 | 114.2 | 124.9 | 147.9 | 150.3 | 240.6 | 244.9 | 263.0 | 430.2 |
| 64 | 97.6 | 87.4 | 89.3 | 115.0 | 128.9 | 151.8 | 212.3 | 224.5 |
| Mean | 199.3 | 295.8 | 336.6 | 409.1 | 425.8 | 499.7 | 543.1 | 666.3 |
| se | 23.9 | 42.7 | 49.2 | 65.6 | 55.1 | 71.1 | 79.1 | 89.1 |
| Doxorubicin | Days from inoculum | | | | | | | |
| # | 50 | 54 | 58 | 62 | 65 | 69 | 72 | 75 |
| 25 | 561.6 | 776.7 | 819.5 | 955.7 | 960.1 | 1238.0 | 1440.5 | 1747.2 |
| 57 | 1087.6 | 1060.5 | 1324.9 | 1686.9 | 1836.1 | 2045.0 | 2313.3 | 2786.0 |
| 65 | 1194.0 | 1306.3 | 1508.4 | 1777.9 | 2144.7 | 2383.0 | 2810.6 | 3155.1 |
| 61 | 996.7 | 1175.7 | 1392.3 | 1604.2 | 1949.8 | 2019.6 | 2245.9 | 2700.8 |
| 12 | 680.6 | 807.3 | 998.2 | 1087.3 | 1415.2 | 1526.2 | 1770.1 | 2034.9 |
| 21 | 642.9 | 620.9 | 733.0 | 777.9 | 837.9 | 909.3 | 1114.8 | 1086.0 |
| 62 | 583.1 | 693.3 | 855.0 | 1128.3 | 1390.9 | 1605.4 | 1904.1 | 2058.0 |
| 8 | 405.8 | 465.0 | 563.7 | 667.3 | 744.7 | 834.8 | 903.5 | 1112.1 |
| 64 | 292.0 | 387.3 | 428.2 | 471.1 | 532.1 | 860.3 | 908.8 | 1057.2 |
| Mean | 716.0 | 810.3 | 958.1 | 1128.5 | 1312.4 | 1491.3 | 1712.4 | 1970.8 |
| se | 103.4 | 104.8 | 126.1 | 156.3 | 192.6 | 191.0 | 224.3 | 264.0 |

† sacrificed moribund

TABLE 5
Antitumour activity of EDO-S101 against DD013 dedifferentiated
liposarcoma xenograft: statistical analysis

| | Days from inoculum | | | | |
|---|---|---|---|---|---|
| | 36 | 40 | 43 | 47 | 50 |
| Vehicles vs. EDO 80 | p < 0.05 | p < 0.0001 | p < 0.0001 | p < 0.0001 | p < 0.0001 |
| Vehicles vs. EDO60 | p < 0.01 | p < 0.0001 | p < 0.0001 | p < 0.0001 | p < 0.0001 |
| Vehicles vs. EDO40 | ns | p < 0.001 | p < 0.0001 | p < 0.0001 | p < 0.0001 |
| Vehicles vs. Doxorubicin | ns | p < 0.01 | p < 0.0001 | p < 0.0001 | p < 0.0001 |

TABLE 6
Mouse body weight (g) during the in vivo evaluation of EDO-S101 in DD013 dedifferentiated
liposarcoma xenograft (treatments started on day 23).

| Vehicles | Days from inoculum | | | | | | | | |
|---|---|---|---|---|---|---|---|---|---|
| # | 23 | 26 | 29 | 33 | 36 | 40 | 43 | 47 | 50 |
| 5 | 22.9 | 22.7 | 23.1 | 24.2 | 25.6 | 25.7 | 26.1 | 26.6 | 27.7 |
| 20 | 26 | 27.2 | 27.4 | 27.3 | 28.3 | 30.1 | 29.6 | 30 | 30.5 |
| 34 | 21.8 | 21.4 | 22.1 | 22.7 | 23.5 | 24.6 | 24.1 | 25.3 | 24.7 |
| 69 | 24.1 | 25.3 | 27.7 | 25.6 | 26.3 | 26.2 | 26.7 | 27.4 | 28.2 |
| 17 | 20 | 20.3 | 20.2 | 21.5 | 22.3 | 22.4 | 22.6 | 23.6 | 25.4 |
| 11 | 25.3 | 25.8 | 26.9 | 27.5 | 28.1 | 28.7 | 29.2 | 29.2 | 30.6 |
| 16 | 21.6 | 22.2 | 22.7 | 23 | 23.7 | 23.7 | 24 | 24.1 | 25.2 |
| 29 | 21.4 | 22.7 | 22.3 | 23.3 | 23.5 | 24.2 | 23.8 | 23.9 | 24.4 |
| 10 | 23 | 23.6 | 24.5 | 24.4 | 24.8 | 25.3 | 25 | 25.3 | 25 |
| Mean | 22.9 | 23.5 | 24.1 | 24.4 | 25.1 | 25.7 | 25.7 | 26.2 | 26.9 |
| se | 0.6 | 0.7 | 0.9 | 0.7 | 0.7 | 0.8 | 0.8 | 0.8 | 0.8 |
| BWL % | 0.0 | 2.5 | 5.2 | 6.5 | 9.7 | 12.0 | 12.1 | 14.2 | 17.3 |

| EDO40 | Days from inoculum | | | | | | | | | | | |
|---|---|---|---|---|---|---|---|---|---|---|---|---|
| # | 23 | 26 | 29 | 33 | 36 | 40 | 43 | 47 | 50 | 54 | 58 | 62 |
| 3 | 23.3 | 22.7 | 23.3 | 23.5 | 24.1 | 24.3 | 24.4 | 26 | 26.1 | 26 | 25.8 | 25.6 |
| 49 | 24.8 | 24.6 | 26 | 25.3 | 25.7 | 26.9 | 26.5 | 27 | 27.5 | 27.2 | 27.2 | 27.7 |
| 46 | 23.5 | 23.4 | 24.8 | 23.4 | 24.6 | 25.2 | 24.9 | 26.1 | 26.1 | 25.3 | 25.2 | 26 |
| 43 | 25.5 | 23.1 | 25.3 | 24.5 | 25.3 | 25.8 | 25.7 | 26.9 | 26.4 | 26.4 | 25.3 | 27 |
| 36 | 21.7 | 21.8 | 22.9 | 22.9 | 22.7 | 22.8 | 23.5 | 24 | 24.4 | 24.6 | 23.4 | 23.8 |
| 35 | 26 | 25.5 | 27.1 | 26.6 | 26.9 | 28.2 | 27.9 | 28.7 | 28.7 | 29 | 28.7 | 29.6 |
| 44 | 21.6 | 20.8 | 23.2 | 22.8 | 23.3 | 23.2 | 23.2 | 25 | 25.2 | 24.6 | 24.3 | 25.2 |
| 58 | 21.1 | 21 | 22.2 | 21.3 | 21.6 | 21.6 | 21.8 | 22.4 | 23.4 | 22.1 | 21.9 | 21.8 |
| 54 | 25 | 25.9 | 27.4 | 27.2 | 27.1 | 27.4 | 27.8 | 28.3 | 29.6 | 28.7 | 28.4 | 29.2 |
| Mean | 23.6 | 23.2 | 24.7 | 24.2 | 24.6 | 25.0 | 25.1 | 26.0 | 26.4 | 26.0 | 25.6 | 26.2 |
| se | 0.6 | 0.6 | 0.6 | 0.6 | 0.6 | 0.7 | 0.7 | 0.7 | 0.7 | 0.7 | 0.8 | 0.8 |
| BWL % | 0.0 | −1.7 | 4.6 | 2.4 | 4.1 | 6.1 | 6.2 | 10.3 | 11.7 | 10.1 | 8.3 | 11.0 |

| EDO40 | Days from inoculum | | | | | | | | | | |
|---|---|---|---|---|---|---|---|---|---|---|---|
| # | 65 | 69 | 72 | 75 | 79 | 83 | 87 | 90 | 94 | 98 | 100 |
| 3 | 25.9 | 26.7 | 27 | 27 | 27.7 | 28 | 28.4 | 29.6 | 28.8 | 29.1 | 28.7 |
| 49 | 27.9 | 28.9 | 28.9 | 29.3 | 27.8 | 29.5 | 28.8 | 28.4 | 29.1 | 29.6 | 29.2 |
| 46 | 25.7 | 26 | 26.5 | 27.1 | 27.4 | 27.2 | 27 | 27.6 | 26.8 | 27.8 | 27.3 |
| 43 | 25 | 27.3 | 26.3 | 27.9 | 26.5 | 25.8 | 26.1 | 27.3 | 27.2 | 28.2 | 28 |
| 36 | 23.3 | 25.3 | 24.8 | 24.9 | 24.9 | 25 | 24.7 | 25.8 | 25.3 | 24.9 | 24.2 |
| 35 | 29.6 | 30 | 30.3 | 30.6 | 29.7 | 30.5 | 30 | 31 | 30.7 | 31.5 | 30.3 |
| 44 | 24.5 | 25 | 25.6 | 26 | 25.3 | 26.4 | 27 | 27.2 | 27.6 | 27.6 | 26.8 |
| 58 | 21.7 | 22.4 | 23.1 | 23.5 | 23.8 | 22.5 | 22.7 | 23.6 | 22.9 | 22.7 | 22.2 |
| 54 | 28.3 | 29.2 | 28.3 | 29.3 | 28.8 | 27.9 | 28.4 | 30 | 28.2 | 30 | 29.2 |
| Mean | 25.8 | 26.8 | 26.8 | 27.3 | 26.9 | 27.0 | 27.0 | 27.8 | 27.4 | 27.9 | 27.3 |
| se | 0.8 | 0.8 | 0.7 | 0.8 | 0.6 | 0.8 | 0.8 | 0.8 | 0.8 | 0.9 | 0.9 |
| BWL % | 9.1 | 13.3 | 13.3 | 15.6 | 13.8 | 14.3 | 14.4 | 17.9 | 16.0 | 18.3 | 15.7 |

| EDO60 | Days from inoculum | | | | | | | | |
|---|---|---|---|---|---|---|---|---|---|
| # | 23 | 26 | 29 | 33 | 36 | 40 | 43 | 47 | 50 |
| 39 | 22.1 | 22.1 | 23.3 | 22.7 | 22.8 | 22.2 | 22.7 | 24.1 | 24.4 |
| 23 | 22.5 | 21.2 | 22.6 | 21.9 | 22.9 | 21.7 | 22.6 | 23.9 | 24.2 |
| 7 | 24 | 23.1 | 24.6 | 24.8 | 24.8 | 24.8 | 26.1 | 26.3 | 26.1 |
| 40 | 22.8 | 21.7 | 22.5 | 22.2 | 23.2 | 22.5 | 23.5 | 24.2 | 24 |
| 53 | 23.2 | 22.6 | 23.6 | 23.8 | 24 | 23.8 | 24.4 | 25.3 | 25.5 |
| 42 | 22.9 | 21.7 | 23.4 | 21.9 | 23 | 21.6 | 23.1 | 27 | 25.4 |
| 68 | 23.8 | 23 | 24.8 | 24.1 | 25.2 | 24.4 | 25.7 | 25.2 | 25.6 |
| 52 | 25.3 | 23 | 24.9 | 24.6 | 25.9 | 25.3 | 26.1 | 27.1 | 27.4 |
| 51 | 23.4 | 22.2 | 23.8 | 22.3 | 23.4 | 22.5 | 23.1 | 23.8 | 24.8 |
| Mean | 23.3 | 22.3 | 23.7 | 23.1 | 23.9 | 23.2 | 24.1 | 25.2 | 25.3 |
| se | 0.3 | 0.2 | 0.3 | 0.4 | 0.4 | 0.5 | 0.5 | 0.4 | 0.4 |
| BWL % | 0.0 | −4.5 | 1.7 | −0.8 | 2.5 | −0.6 | 3.5 | 8.0 | 8.3 |

| EDO60 | Days from inoculum | | | | | | | | | |
|---|---|---|---|---|---|---|---|---|---|---|
| # | 54 | 58 | 62 | 65 | 69 | 72 | 75 | 79 | 83 | 87 |
| 39 | 25.5 | 24.5 | 24.4 | 25.1 | 26 | 27.1 | 25.7 | 25 | 25.6 | 25.3 |
| 23 | 23.8 | 24.1 | 23.8 | 23.5 | 24.1 | 24.4 | 25.1 | 23 | 23.6 | 24.1 |
| 7 | 27.4 | 26.5 | 27 | 27.4 | 27.2 | 28.2 | 26.1 | 25.9 | 25.4 | 26.9 |
| 40 | 25.5 | 24.8 | 25.2 | 25.8 | 25.5 | 25.8 | 25.5 | 24.6 | 23.8 | 24.3 |
| 53 | 26.5 | 25.6 | 26 | 26.6 | 26.1 | 26.4 | 26.1 | 25.4 | 26.1 | 25.9 |
| 42 | 24.4 | 24.8 | 24.7 | 24.8 | 25.3 | 25.6 | 25.7 | 24.1 | 23.7 | 24.8 |
| 68 | 25.7 | 26.7 | 25.8 | 25.7 | 25.3 | 26.1 | 27.1 | 25.9 | 25.6 | 25 |
| 52 | 26.8 | 27.6 | 27.7 | 27 | 27.2 | 26.9 | 28.6 | 26 | 26.1 | 24.6 |
| 51 | 24.6 | 25.3 | 25.1 | 25.6 | 26.5 | 26.2 | 24.5 | 23.2 | 21 | 20.6 |
| Mean | 25.6 | 25.5 | 25.5 | 25.7 | 25.9 | 26.3 | 26.0 | 24.8 | 24.5 | 24.6 |
| se | 0.4 | 0.4 | 0.4 | 0.4 | 0.3 | 0.4 | 0.4 | 0.4 | 0.6 | 0.6 |
| BWL % | 9.6 | 9.5 | 9.4 | 10.2 | 11.0 | 12.7 | 11.6 | 6.2 | 5.2 | 5.5 |

| EDO60 | Days from inoculum | | | | | | | | |
|---|---|---|---|---|---|---|---|---|---|
| # | 90 | 94 | 98 | 100 | 103 | 107 | 111 | 114 | 118 |
| 39 | 25.9 | 26.4 | 27.2 | 26.5 | 25.1 | 25.6 | 21.4 | 21.3 | 21.9 |
| 23 | 23.8 | 23.7 | 25.3 | 25.3 | 25.8 | 25.6 | 26 | 25.5 | 26 |
| 7 | 26.9 | 27 | 28.1 | 27.1 | 26.8 | 26 | 23.8 | 26.2 | 25.6 |
| 40 | 24.7 | 25.5 | 26.6 | 25.7 | 26.4 | 26.4 | 27.2 | 26.3 | 26.3 |
| 53 | 26.8 | 26.2 | 27.3 | 26.3 | 27.2 | 27.2 | 27.8 | 27.5 | 26.8 |
| 42 | 24.7 | 24.5 | 25.1 | 24.4 | 25 | 25.5 | 26.2 | 25.6 | 25 |
| 68 | 24.5 | 23.8 | 24.5 | 23.9 | 23.6 | 24.9 | 26.2 | 25.8 | 27.3 |
| 52 | 25.5 | 28.5 | 28.9 | 28.9 | 29 | 28.3 | 28.8 | 28 | 29.5 |
| 51 | 20.7 | 21.7 | 22.6 | 23.2 | 22.5 | 22.1 | 22.8 | 21.7 | 20.8 |
| Mean | 24.8 | 25.3 | 26.2 | 25.7 | 25.7 | 25.7 | 25.6 | 25.3 | 25.5 |
| se | 0.6 | 0.7 | 0.7 | 0.6 | 0.6 | 0.6 | 0.8 | 0.8 | 0.9 |
| BWL % | 6.4 | 8.2 | 12.2 | 10.1 | 10.2 | 10.3 | 9.6 | 8.5 | 9.1 |

| EDO80 | Days from inoculum | | | | | | | | | | | |
|---|---|---|---|---|---|---|---|---|---|---|---|---|
| # | 23 | 26 | 29 | 33 | 36 | 40 | 43 | 47 | 50 | 54 | 58 | 62 |
| 33 | 26.1 | 23.4 | 22.7 | 25.1 | 28.3 | 28.6 | 28.5 | 28.4 | 27.9 | 27.7 | 28.7 | 25.7 |
| 56 | 25.7 | 23.6 | 22.6 | 25.1 | 27 | 27.2 | 26.5 | 27.9 | 28.3 | 28.4 | 29.8 | 24.4 |
| 32 | 25 | 22 | 21.5 | 24 | 25.3 | 27 | 26 | 26.1 | 26.8 | 26.7 | 28 | 23.9 |
| 60 | 23.1 | 21.3 | 22.2 | 23.3 | 25.6 | 25.7 | 25 | 25.4 | 25.4 | 25.8 | 27.4 | 24 |
| 59 | 24 | 20.9 | 21 | 23.1 | 24.7 | 26 | 25.6 | 24.6 | 24.6 | 25 | 26.9 | 22.7 |
| 14 | 23.3 | 20.1 | 21 | 21.9 | 23.8 | 24.4 | 25 | 24.2 | 24.4 | 24.5 | 25 | 21.9 |
| 50 | 26.2 | 22.4 | 21.6 | 24.9 | 26.3 | 27.9 | 26.9 | 27.4 | 26.3 | 28 | 27.6 | 23.7 |
| 37 | 22.4 | 21.6 | 21.6 | 22.5 | 24.2 | 24 | 24.2 | 24.6 | 23.2 | 24.8 | 24.9 | 23.8 |
| 38 | 22.3 | 21.9 | 21 | 23.3 | 24.6 | 24.4 | 24.4 | 24.4 | 25.3 | 25 | 25 | 21.4 |
| Mean | 24.2 | 21.9 | 21.7 | 23.7 | 25.5 | 26.1 | 25.8 | 25.9 | 25.8 | 26.2 | 27.0 | 23.5 |
| se | 0.5 | 0.4 | 0.2 | 0.4 | 0.5 | 0.6 | 0.5 | 0.5 | 0.6 | 0.5 | 0.6 | 0.4 |
| BWL % | 0.0 | −3.4 | 4.2 | 1.3 | 5.9 | 2.5 | 8.0 | 5.9 | 7.6 | 8.0 | 12.2 | 8.4 |

| EDO80 | Days from inoculum | | | | | | | | | | | | |
|---|---|---|---|---|---|---|---|---|---|---|---|---|---|
| # | 65 | 69 | 72 | 75 | 79 | 83 | 87 | 90 | 94 | 98 | 100 | 103 | 107 |
| 33 | 27.2 | 28.7 | 30.7 | 30.6 | 28.7 | 24.1 | 25.3 | 29.2 | 31 | 31.1 | 29.7 | 25.9 | 26 |
| 56 | 24.1 | 25.1 | 23.1 | 21.8 | 16.6 | † | | | | | | | |
| 32 | 24.7 | 25.8 | 27.5 | 27.4 | 26.2 | 21.1 | 23.9 | 28 | 29.1 | 29.9 | 28.6 | 28.3 | 23.3 |
| 60 | 25.2 | 26 | 26.5 | 27.4 | 26.3 | 26.8 | 27.2 | 28.2 | 28.9 | 28 | 27.6 | 28.4 | 28.4 |
| 59 | 23.1 | 24.4 | 26.5 | 27.2 | 24.6 | 25 | 20.4 | 18.3 | 23.3 | 27.6 | 27.3 | 26.7 | 24.5 |
| 14 | 22.8 | 24.1 | 25.2 | 26.6 | 24.1 | 26.3 | 24.8 | 26.1 | 25.9 | 26.1 | 26.1 | 27.1 | 25.4 |
| 50 | 24.1 | 26.4 | 25.5 | 27.4 | 27.6 | 27.4 | 27.1 | 24.4 | 28.5 | 28.6 | 28.1 | 28.2 | 27.5 |
| 37 | 23.1 | 23.9 | 25 | 24.8 | 26 | 25.3 | 24.2 | 26 | 26 | 24.6 | 25.1 | 25.4 | 26.4 |
| 38 | 21.1 | 22.3 | 23.4 | 24.3 | 21.3 | 17.1 | 17.2 | 23.1 | 26.7 | 28 | 27.7 | 28.4 | 27.6 |
| Mean | 23.9 | 25.2 | 25.9 | 26.4 | 24.6 | 24.1 | 23.8 | 25.4 | 27.4 | 28.0 | 27.5 | 27.3 | 26.1 |
| se | 0.6 | 0.6 | 0.8 | 0.8 | 1.2 | 1.2 | 1.2 | 1.2 | 0.9 | 0.7 | 0.5 | 0.4 | 0.6 |
| BWL % | 8.0 | 6.3 | 9.7 | 13.9 | 8.8 | 7.6 | 5.0 | 2.9 | 0.0 | 2.9 | 0.4 | −0.8 | 4.6 |

| Doxo | Days from inoculum | | | | | | | |
|---|---|---|---|---|---|---|---|---|
| # | 23 | 26 | 29 | 33 | 36 | 40 | 43 | 47 |
| 25 | 25.7 | 25.6 | 26.1 | 24.6 | 25.3 | 25 | 25.6 | 26.9 |
| 57 | 25.2 | 24.7 | 24.8 | 24.4 | 24.2 | 25.3 | 26.3 | 27 |
| 65 | 24 | 23.8 | 25.4 | 23.5 | 24.8 | 25.3 | 26.4 | 27 |
| 61 | 22.8 | 22.7 | 22.7 | 22.2 | 23.4 | 23.7 | 24.4 | 25.7 |
| 12 | 23 | 22.7 | 23.1 | 22 | 22.8 | 24.4 | 24.9 | 26.2 |
| 21 | 23.6 | 22.7 | 22.7 | 21.4 | 21 | 21.9 | 23.7 | 25.3 |
| 62 | 24.5 | 23.1 | 24.1 | 23 | 24 | 24.3 | 24.6 | 25.7 |
| 8 | 23.6 | 22.7 | 23.1 | 22.3 | 23.8 | 24.4 | 24.6 | 25.4 |
| 64 | 20.6 | 19.4 | 20.4 | 18.5 | 20 | 20.1 | 21 | 21.7 |
| Mean | 23.7 | 23.0 | 23.6 | 22.4 | 23.3 | 23.8 | 24.6 | 25.7 |
| se | 0.5 | 0.6 | 0.6 | 0.6 | 0.6 | 0.6 | 0.5 | 0.5 |
| BWL % | 0.0 | −2.6 | −0.3 | −5.2 | −1.7 | 0.7 | 4.0 | 8.4 |
| Doxo | Days from inoculum | | | | | | | |
| # | 50 | 54 | 58 | 62 | 65 | 69 | 72 | 75 |
| 25 | 25.9 | 25.8 | 26.1 | 26.8 | 24 | 26.6 | 26.5 | 26.9 |
| 57 | 26.8 | 26.6 | 26.9 | 27 | 27 | 27.9 | 27.3 | 27.8 |
| 65 | 27 | 26.1 | 26 | 27.3 | 27.7 | 29.1 | 27 | 25.9 |
| 61 | 24.2 | 24.9 | 25 | 26.2 | 25.6 | 27.4 | 26.7 | 27.1 |
| 12 | 25.3 | 25 | 25.2 | 26.2 | 26.2 | 26.8 | 27.4 | 27 |
| 21 | 24.4 | 23.7 | 23.8 | 24.1 | 24.5 | 25.1 | 25.1 | 25 |
| 62 | 23.9 | 25.9 | 25.7 | 26.1 | 26.2 | 27.6 | 26.5 | 27 |
| 8 | 25.2 | 25.4 | 25.3 | 26.1 | 26.1 | 27 | 26.8 | 26.1 |
| 64 | 21.2 | 21.3 | 21.3 | 22.2 | 21.5 | 22.2 | 21.1 | 22.2 |
| Mean | 24.9 | 25.0 | 25.0 | 25.8 | 25.4 | 26.6 | 26.0 | 26.1 |
| se | 0.6 | 0.5 | 0.5 | 0.5 | 0.6 | 0.7 | 0.7 | 0.6 |
| BWL % | 5.1 | 5.5 | 5.8 | 8.9 | 7.4 | 12.5 | 10.0 | 10.3 |

† sacrificed moribund

Comparing Example 1 and Example 2, EDO-S101 appears to be better tolerated in DD013 xenograft models, compared with TC-71 xenograft models. EDO-S101 was shown to be very effective against liposarcoma. The dose of 60 mg/kg administered q7dx3 resulted to be the best choice both in terms of efficacy and tolerability.

### Example 3

A Phase 1/2 Study to Investigate the Safety, Pharmacokinetics and Efficacy of EDO-S101, a First-In-Class Alkylating Histone Deacetylase Inhibition (HDACi) Fusion Molecule, in Patients with Advanced Solid Tumours

### Purpose

Phase 1: To determine the safety, tolerability, maximum tolerated dose (MTD), and recommended phase 2 dose (RP2D) of EDO-S101 as a single agent in patients with solid tumours who have progressed after at least one (1) line of standard therapy.

Phase 2: To evaluate the efficacy of EDO-S101 in selected tumour types.

| Condition | Intervention | Phase |
|---|---|---|
| Phase 1: | Drug: EDO-S101 | Phase 1/2 |
| Advanced or metastatic Solid Tumours | | |
| Phase 2: | | |
| small cell lung cancer (SCLC) | | |
| soft tissue sarcoma or non-Kit GIST | | |
| triple negative breast cancer | | |
| ovarian cancer | | |

Study Type: Interventional
Study Design: Intervention Model: Single Group Assignment
Masking: Open Label
Primary Purpose: Treatment
Primary Outcome Measures:

### Phase 1: Dose Escalation Until Maximum Administered Dose (MAD):

Determine Maximum Tolerated Dose at optimal infusion schedule

### Phase 2: Evaluation of Toxicity and Response Rate in Selected Solid Tumour Cohorts:

Confirm recommended Phase 2 dose and optimal infusion schedule in select solid tumours
Determine objective response rate and clinical benefit rate in selected solid tumours

### Secondary Outcome Measures:

### Phase 1: Dose Escalation Until MAD:

Determine maximum plasma concentration (Cmax) of EDO-S101

### Phase 2: Evaluation of Toxicity and Response Rate in Selected Solid Tumour Cohorts:

To evaluate safety and tolerability of the RP2D of the selected schedule of EDOS101.
To determine the progression free survival time for patients who received the RP2D at the optimal infusion schedule
To determine overall survival for patients who received the RP2D at the selected study drug administration schedule.
To establish the trough PK profiles of EDO-S101.

### Estimated Enrollment: 158

### Phase 1:

Schedule A: EDO-S101, IV, 60 mg/m² up to 150 mg/m² Day 1 and 15 of each 28 day cycle Schedule B: EDO-S101, IV, 60 mg/m² up to 150 mg/m² Day 1, 8 and 15 of each 28 day cycle

### Phase 2:

The RP2D and selected schedule will be further investigated in patients with specific types of solid tumours: relapsed/refractory SCLC, soft tissue sarcoma, non-Kit GIST, triple negative breast, and ovarian cancers.

### DETAILED DESCRIPTION

EDO-S101 I entity, a first-in-class fusion molecule of an alkylator, bendamustine and a histone-deacetylase inhibitor (HDACi), vorinostat. This phase 1/2 study will enroll patients with solid tumours. This phase 1/2 study will enroll patients with various advanced solid tumours.

The study consists of 2 phases:
Phase 1: Dose Escalation until MAD
Phase 2: Evaluation of Toxicity and Response Rate in Selected Solid Tumour Cohorts

The study is designed as an open label, Phase 1/2 trial of single agent EDOS101. The phase 1 portion of the study is designed to define the MTD for two (2) administration schedules by evaluating toxicities during dose escalation until MAD. The phase 2 portion of the study is designed to evaluate ORR and CBR at four (4) or six (6) months depending on the type of solid tumour.

### Eligibility

Ages Eligible for Study: 18 Years and older (Adult, Senior)
Sexes Eligible for Study: All
Accepts Healthy Volunteers: No

### Criteria

### Inclusion Criteria for Phase 1 and Phase 2 Portions of Study:

1. Signed informed consent.
2. patients≥18 years at signing the informed consent.
3. Diagnosis of advanced or metastatic solid tumours, disease should have progressed following at least one line of standard therapy.
4. Patients with secondary metastasis to the CNS are eligible if they have met certain criteria.
5. Evaluable disease; either measurable on imaging or with informative tumour marker.
6. Discontinuation of previous cancer therapies at least three (3) weeks or 5 half-lives, whichever is shorter.
7. Eastern Cooperative Oncology Group (ECOG) performance status≤2
8. Neutrophils≥1,500 μL.
9. Platelets≥100,000 μL.
10. Aspartate aminotransferase/alanine aminotransferase (AST/ALT)≤3 upper limit of normal (ULN). In cases with liver involvement ALT/AST≤5×ULN.
11. Total bilirubin: 51.5 mg/dL unless elevated due to known Gilbert's syndrome.
12. Creatinine≤1.5 ULN.
13. Serum potassium within normal range.
14. If female of child-bearing potential (i.e. not postmenopausal or surgically sterile), must be willing to abstain from sexual intercourse or employ an effective barrier or medical method of contraception during the study drug administration and follow-up periods. If male, must be sterile or willing to abstain from sexual intercourse or employ a barrier method of contraception during the study treatment and at least 6 months following last treatment.

### Exclusion Criteria for Phase 1 and Phase 2 Portions of Study:

1. Patients with primary central nervous system (CNS) cancer.
2. Patients with QTc interval>450 msec for male and >470 msec for female.
3. Patients who are on treatment with drugs known to prolong the QT/QTc interval.
4. Patients who are on treatment with Valproic Acid in any of its indication (epilepsy, mood disorder) must be excluded or must stop using the medication.
5. Any serious medical condition that interferes with adherence to study procedures.
6. Prior history of solid tumour malignancy diagnosed within the last three (3) years of study enrollment excluding adequately treated basal cell carcinoma of the skin, squamous cell carcinoma of the skin, or in situ cervical cancer, in situ breast cancer, in situ prostate cancer (patients must have shown no evidence of active disease for 2 years prior to enrollment)
7. Pregnant or breast feeding females.
8. New York Heart Association (NYHA) stage Ill/IV congestive heart failure, arrhythmias not adequately controlled, or other significant co-morbidities [e.g. active infection requiring systemic therapy, history of human immunodeficiency virus (HIV) infection, or active Hepatitis B or Hepatitis C].
9. Use of other investigational agents within 30 days or 5 half-lives prior to the first dose of study drug. As long as patient has recovered from any related toxicities Grade 1.
10. Steroid treatment within seven (7) days prior to study treatment. Patients that require intermittent use of bronchodilators, topical steroids or local steroid injections will not be excluded from the study. Patients who have been stabilized to 10 mg orally prednisolone PO QD (or equivalent), daily or less seven (7) days prior to study drug administration are allowed.

Phase 2 Tumour-specific Eligibility Criteria

Phase 2 patients must meet the cohort-specific inclusion/exclusion criteria in addition to the general inclusion/exclusion criteria for Phase 1 and Phase 2 study listed above.

### Cohort 1 Patient Population: Relapsed/Refractory SCLC

1. Histologically or cytological confirmed limited or extensive disease stage of SCLC. The disease should be progressing during or relapsing after the previous treatment.
2. At least one line of prior combination chemotherapy including adequate doses of platinum compound and having progressed during therapy or after the previous treatment.
3. At least 3 weeks or 5 half-lives, whichever is shorter, should have elapsed since prior treatment as long as the patient recovered from any related toxicities to ≤Grade 1.
4. Prior radiotherapy is acceptable provided the patient has recovered from any radiotherapy related acute toxicities.

### Cohort 2 Patient Population: Relapsed/Refractory Soft Tissue Sarcoma or Non-Kit GIST

1. Histologically confirmed diagnosis of advanced, unresectable, or metastatic soft tissue sarcoma not amenable to curative treatment with surgery or radiotherapy excluding: chondrosarcoma, neuroblastoma, osteosarcoma, embryonal rhabdomyosarcoma, or Kaposi sarcoma.
2. Must have received at least one prior first line combination chemotherapy regimen or at least two first line single-agent regimens. Adjuvant chemotherapy not considered first line, unless disease progression within 6 months of treatment.
3. The disease should be progressing/relapsed during or after the previous treatment. At least 3 weeks should have elapsed since prior chemotherapy or 5 half-lives, whichever is shorter, as long as the patient recovered from any related toxicities to ≤Grade 1.
4. Presence of measurable disease as defined by the Response Evaluation Criteria in Solid Tumours (RECIST 1.1, Eisenhauer et al. 2009).

### Cohort 3 Patient Population: Relapsed/Refractory Triple Negative Breast Cancer

1. Histologically or cytologically confirmed locally advanced or metastatic Triple Negative Metastatic Breast Cancer.
2. Must have received at least one line of chemotherapy, at least 3 weeks should have relapsed since prior chemotherapy or 5 half-lives, whichever is shorter, as long as the patient recovered from acute toxicity of previous therapies to ≤grade 1.
3. Prior radiotherapy is acceptable provided it was applied within 4 four weeks before starting of this trial and the patient recovered from any radiotherapy related acute toxicities.
4. The disease should be progressing/relapsed during or after the previous treatment.
5. Presence of measurable disease as defined by the Response Evaluation Criteria.

### Cohort 4 Patient Population: Relapsed/Refractory Ovarian Cancer

1. Histologically or cytologically confirmed advanced ovarian cancer: epithelial ovarian cancer, primary peritoneal cancer or fallopian tube cancer (excluding borderline ovarian cancer) that is resistant or refractory to platinum therapy.
   a. Platinum-resistant ovarian cancer is defined as disease that responded to primary platinum therapy and then progressed within 6 months or disease that progressed during or within six months of completing a subsequent platinum therapy.
   b. Primary platinum refractory disease is defined as disease that has not responded to a platinum-based regimen or experienced disease recurrence within 3 months of completing a first-line platinum-based regimen.
2. The disease should be progressing/relapsed during or after the previous treatment. At least 3 weeks should have elapsed since prior chemotherapy or 5 half-lives, whichever is shorter, as long as the patient recovered from acute toxicity of previous therapies to ≤grade 1.
3. Presence of measurable disease as defined by the Response Evaluation Criteria in Solid tumours (RECIST 1.1, Eisenhauer et al. 2009).

1. Tinostamustine or a pharmaceutically acceptable salt thereof for use in the treatment of sarcoma in a patient in need thereof selected from soft tissue sarcoma, bone sarcoma or non-KIT gastrointestinal stromal tumour (GIST).

2. Tinostamustine or a pharmaceutically acceptable salt thereof for use in the treatment of sarcoma according to claim 1 wherein the sarcoma is from the Ewing family of tumours.

3. Tinostamustine or a pharmaceutically acceptable salt thereof for use in the treatment of sarcoma according to claim 2 wherein the sarcoma is Ewing tumour of bone.

4. Tinostamustine or a pharmaceutically acceptable salt thereof for use in the treatment of sarcoma according to claim 2 wherein the sarcoma is extraosseous Ewing tumour.

5. Tinostamustine or a pharmaceutically acceptable salt thereof for use in the treatment of sarcoma according to claim 2 wherein the sarcoma is primitive neuroectodermal tumour (PNET).

6. Tinostamustine or a pharmaceutically acceptable salt thereof for use in the treatment of sarcoma according to claim 1 wherein the sarcoma is liposarcoma.

7. Tinostamustine or a pharmaceutically acceptable salt thereof for use in the treatment of sarcoma according to claim 6 wherein the liposarcoma is well-differentiated liposarcoma, myxoid liposarcoma, pleomorphic liposarcoma, or dedifferentiated liposarcoma.

8. Tinostamustine or a pharmaceutically acceptable salt thereof for use in the treatment of sarcoma according to claim 6 wherein the liposarcoma is dedifferentiated liposarcoma.

9. Tinostamustine or a pharmaceutically acceptable salt thereof for use in the treatment of sarcoma according to any preceding claim, wherein the cancer is relapsed and/or refractory.

10. Tinostamustine or a pharmaceutically acceptable salt thereof for use in the treatment of sarcoma according to any preceding claim, wherein tinostamustine or a pharmaceutically acceptable salt thereof is administered intravenously to the patient in need thereof at a dosage level of from 0.3 to 300 mg/m² body surface area of the patient, of from 60 to 150 mg/m² body surface area of the patient, or of from 80 to 100 mg/m² body surface area of the patient.

11. Tinostamustine or a pharmaceutically acceptable salt thereof for use in the treatment of sarcoma according to any one of claims 1 to 10, wherein tinostamustine or a pharmaceutically acceptable salt thereof is administered intravenously to the patient in need thereof on days 1, 8 and 15 of a 28 day treatment cycle, or on days 1 and 15 of a 28 day treatment cycle.

12. Tinostamustine or a pharmaceutically acceptable salt thereof for use in the treatment of sarcoma according to any one of claims 1 to 11, wherein tinostamustine or a pharmaceutically acceptable salt thereof is administered intravenously to the patient in need thereof over an infusion time of 60 minutes; or an infusion time of 45 minutes; or an infusion time of 30 minutes.

13. Tinostamustine or a pharmaceutically acceptable salt thereof for use in the treatment of sarcoma according to any one of claims 1 to 12, wherein tinostamustine or a pharmaceutically acceptable salt thereof is administered intravenously to the patient in need thereof at a dosage level of from 80 mg/m² to 100 mg/m² body surface area of the patient, on days 1 and 15 of a 28 day treatment cycle, and over an infusion time of 60 minutes.

14. Tinostamustine or a pharmaceutically acceptable salt thereof for use in the treatment of sarcoma according to any one of claims 1 to 13, wherein the patient is treated with tinostamustine or a pharmaceutically acceptable salt thereof and radiotherapy

15. Tinostamustine or a pharmaceutically acceptable salt thereof for use in the treatment of sarcoma according to claim 14, wherein said radiotherapy treatment is given to the patient in need thereof at a dose of 1 to 5 Gy over 5-10 consecutive days, and preferably 2 Gy over 5-10 consecutive days.

16. A kit comprising tinostamustine or a pharmaceutically acceptable salt thereof together with instructions for treating sarcoma selected from soft tissue sarcoma, bone sarcoma or non-KIT gastrointestinal stromal tumour (GIST).
